SEQUENCE LISTING (1 sequence)

SEQ ID NO: 1 - protein, 19 aa, Artificial Sequence
  synthetic peptide
GERRQYFWIAWYKLANSKK

(19) United States
(12) Patent Application Publication
(10) Pub. No.: US 2021/0060125 A1
(43) Pub. Date: Mar. 4, 2021

(54) METHOD AND USE OF PnPP-19 FOR PREVENTING AND TREATING EYE DISEASES

(71) Applicants: Biozeus Desenvolvimento De Produtos Biofarmacêuticos, Rio de Janeiro (BR); Universidade Federal de Minas Gerais, Belo Horizonte (BR)

(72) Inventors: Armando da Silva Cunha Junior, Belo Horizonte (BR); Maria Elena de Lima Perez Garcia, Belo Horizonte (BR); Carolina Nunes da Silva, Belo Horizonte (BR); Lays Fernanda Nunes Dourado, Belo Horizonte (BR); Perla Villani Borges da Silva, Rio de Janeiro (BR); Paulo Gustavo Sampaio Lacativa, Rio de Janeiro (RJ); Gerhard Max Gross, Fuldabrück (DE); Iron Francisco de Paula Junior, Rio de Janeiro (BR)

(21) Appl. No.: 17/008,794

(22) Filed: Sep. 1, 2020

Related U.S. Application Data
(60) Provisional application No. 62/895,252, filed on Sep. 3, 2019.

(51) Int. Cl. A61K 38/17; A61P 27/06

(52) U.S. Cl. CPC A61K 38/1767; A61P 27/06

(57) ABSTRACT

The present description relates to methods of treatment and pharmaceutical compositions comprising a Nitric Oxide Synthase-inducer peptide, PnPP-19. Although usable for other purposes, the composition comprising PnPP-19 is useful for treating and/or preventing eye diseases related to intraocular hypertension and/or optic nerve degeneration, such as glaucoma.

### CROSS-REFERENCE TO RELATED APPLICATIONS

This application claims the benefit of priority from U.S. Provisional Application No. 62/895,252, filed Sep. 3, 2019, the entire content of which is incorporated herein by reference.

### BACKGROUND

Glaucoma represents a heterogeneous group of chronically progressive optic neuropathies. It is the leading cause of irreversible blindness worldwide, affecting around 64 million people globally. This figure is projected to increase to 80 million in 2020 and 112 million by 2040 (THAM, Y. C., LI, X., WONG, T. Y., QUIGLEY, H. A., AUNG, T., CHENG, C. Y. Global prevalence of glaucoma and projections of glaucoma burden through 2040: a systematic review and meta-analysis. Ophthalmology, 121(11): 2081-2090, 2014; ALIANCY, J., STAMER, W. D., WIROSTKO, B. A Review of Nitric Oxide for the Treatment of Glaucomatous Disease. Ophthalmol Ther, 6(2): 221-232, 2017).

Glaucoma is generically described as a slow degeneration of retinal ganglion cells (RGC) followed by axon loss. It manifests as a progressive thinning of the retinal nerve fiber layer, and optic nerve head cupping, events which functionally result in a characteristic pattern of visual field loss. Optic nerve head cupping is the enlargement of the central portion of the optic disc, which is called the “cup”, a whitish portion without nerve fibers which is usually quite small in comparison with the entire optic disc. The diameter of the cup in relation to the total diameter of the optic disc is called the cup-to-disc ratio, a measurement used to assess the progression of glaucoma. The normal cup-to-disc ratio is 0.3, with higher values indicating pathologies. However it's the increase in cupping as the patient ages that indicates pathology, rather than high, but stable cup-to-disc ratios, that can happen due to hereditary factors even in the absence of glaucoma (WEINREB, R. N., AUNG, T., MEDEIROS, F. A. The pathophysiology and treatment of glaucoma: a review. JAMA, (18):1901-11, 2014).

In glaucoma patients, the nerve fibers begin to die due to increased pressure in the eye and/or loss of blood flow to the optic nerve. The rate of RGC death correlates with the level of intraocular pressure (IOP), which results from a delicate balance in the production and elimination of the aqueous humor (AH) in the anterior eye segment. The AH is produced and secreted by the ciliary epithelium in the posterior eye chamber. Under physiological conditions, the AH passes from the posterior to the anterior chamber through the pupil and is drained from the eye through the conventional (or trabecular) outflow, or through the unconventional (or uveoscleral) outflow pathways (BRAUNGER, B. M., FUCHSHOFER, R., TAMM, E. R. The aqueous humor outflow pathways in glaucoma: A unifying concept of disease mechanisms and causative treatment. Eur J Pharm Biopharm, 95(Pt B):173-81, 2015).

The conventional outflow system comprises the trabecular meshwork (TM), the juxtacanalicular tissue (JCT), and the Schlemm's canal (SC) in humans, non-human primates, and rats (MORRISON, J. C., FRAUNFELDER, F. W., MILNE, S. T., MOORE, C. G. Limbal microvasculature of the rat eye. Invest Ophthalmol Vis Sci, 36: 751-756, 1995; MORRISON, J. C., CEPURNA, W. O., JOHNSON, E. C. Modeling glaucoma in rats by sclerosing aqueous outflow pathways to elevate intraocular pressure, Exp Eye Res, 141: 23-32, 2015). Under physiological conditions, the conventional outflow mediates about 60%-90% of the AH drainage in humans and non-human primates, while the unconventional pathway plays a smaller role, which tends to become even smaller in older eyes (NATHANSON, J. A., MCKEE, M. Identification of an extensive system of nitric oxide-producing cells in the ciliary muscle and outflow pathway of the human eye. Invest Ophthalmol Vis Sci, 36: 1765-1773, 1995). In mice, the unconventional outflow plays a much larger role, accounting for approximately 80% of the AH drainage (AIHARA, M., LINDSEY, J. D., WEINREB, R. N. Aqueous humor Dynamics in Mice. Invest Ophthalmol Vis Sci, 44(12): 5168-73, 2003). Passage of AH in the trabecular outflow pathways is driven by a pressure gradient only. Therefore, increased resistance to the passage of the AH through the conventional system is the key cause of the elevation of IOP above the normal threshold of 20 mmHg (CAVET, M. E., VITTITOW, J. L., IMPAGNATIELLO, F., ONGINI, E., BASTIA, E. Nitric oxide (NO): an emerging target for the treatment of glaucoma. Invest Ophthalmol Vis Sci, 55(8): 5005-5015, 2014; BRAUNGER, B. M., FUCHSHOFER, R., TAMM, E. R. The aqueous humor outflow pathways in glaucoma: A unifying concept of disease mechanisms and causative treatment. Eur J Pharm Biopharm, 95(Pt B):173-81, 2015).

The reduced AH drainage rate by the conventional outflow system is a consequence of abnormal histologic or anatomical changes in particular eye structures, leading to two major groups of glaucoma. In primary angle-closure glaucoma (PACG), different deformations of the iris push it towards the TM, thus blocking the drainage of AH and leading to increased IOP. Cases of PACG account for approximately 25% of the total prevalence of glaucoma (WRIGHT, C., TAWFIK, M. A., WAISBOURD, M., KATZ, L. J. Primary angle-closure glaucoma: an update. Acta Ophthalmol. 94(3): 217-25, 2016).

Another group of the disease—primary open-angle glaucoma (POAG)—is the leading cause of glaucomatous blindness, affecting 80 to 90% of patients with glaucoma. In POAG, histologic alterations of the tissues that constitute the conventional outflow pathway result in the increased resistance to AH drainage through the TM and the SC. There is compelling evidence that increased TM outflow resistance in POAG results from an increased contractile phenotype of the connective tissue of the JCM and the endothelium of the SC (BRAUNGER, B. M., FUCHSHOFER, R., TAMM, E. R. The aqueous humor outflow pathways in glaucoma: A unifying concept of disease mechanisms and causative treatment. Eur J Pharm Biopharm, 95(Pt B): 173-81, 2015).

In one group of individuals, IOP is elevated; however, the symptoms of glaucoma are absent. Nevertheless, it's been shown that the reduction of IOP prevents/delays disease onset in those with high IOP without glaucoma (HEIJL, A. Glaucoma treatment: by the highest level of evidence. Lancet, 385(9975): 1264-1266, 2015; ALIANCY, J., STAMER, W. D., WIROSTKO, B. A Review of Nitric Oxide for the Treatment of Glaucomatous Disease. Ophthalmol Ther, 6(2): 221-232, 2017).

The ultimate goal of anti-glaucoma therapy is to maintain visual function and quality of life of patients. In a subset of patients with POAG, IOP is not elevated, thus characterizing the cases of normal-tension glaucoma (NTG), and patients still develop progressive visual loss. And even when IOP is controlled in patients with POAG, visual loss can still progress. These facts corroborate for the role of loss of blood flow towards the optic nerve in nerve fiber death. Up to now, no drug is able to act on this physiopathology, and therefore all therapeutic options converge to a reduction of IOP, as it is the only modifiable risk factor for disease onset and progression (ALIANCY, J., STAMER, W. D., WIROSTKO, B. A Review of Nitric Oxide for the Treatment of Glaucomatous Disease. Ophthalmol Ther, 6(2): 221-232, 2017). However, managing the disease progression depends on the etiopathology. Cases of PACG are mainly treated with is laser peripheral iridotomy. If IOP is still abnormally high, pharmacological approaches can be used (WEINREB, R. N., AUNG, T., MEDEIROS, F. A. The pathophysiology and treatment of glaucoma: a review. JAMA, (18):1901-11, 2014)).

On the other hand, the treatment of POAG requires pharmacotherapy as soon as glaucoma is diagnosed. The magnitude of the effect of IOP-reducing drugs correlates with the severity of IOP elevation, producing greater reductions in eyes with higher internal pressure. Nevertheless, every unit reduction in mmHg accounts for between 10-19% decrease in the risk of disease progression (HEIJL, A. Glaucoma treatment: by the highest level of evidence. Lancet, 385(9975): 1264-1266, 2015; ALIANCY, J., STAMER, W. D., WIROSTKO, B. A Review of Nitric Oxide for the Treatment of Glaucomatous Disease. Ophthalmol Ther, 6(2): 221-232, 2017).

In general, the initial target aims, for example, at a 20% to 50% reduction in IOP, and it should be achieved with the fewest medications as possible to avoid adverse events. This initial target is valid both for humans as for other animals.

The currently available pharmacological tools reduce IOP either by decreasing AH production and/or improving its outflow. Topic prostaglandin analogs, such as latanoprost, travoprost, tafluprost, unoprostone, bimatoprost, are the first therapeutic option. These drugs act mainly by improving the AH drainage through the uveoscleral, unconventional, pathway. Although less effective, second-line agents are generally used when there is intolerance or contraindication to prostaglandin analogs. Topic alternative drug classes comprise β-Adrenergic and α-Adrenergic blockers, carbonic anhydrase inhibitors and cholinergic agonists. However, when the desired IOP level is not achieved by single therapy, physicians include additional drugs to the treatment schedule (WEINREB, R. N., AUNG, T., MEDEIROS, F. A. The pathophysiology and treatment of glaucoma: a review. JAMA, (18):1901-11, 2014).

Despite the availability of a number of anti-glaucoma medications, there is still a high medical need in this field. Long-term use of IOP-lowering agents may cause, for example, conjunctival hyperemia, uveitis, macular edema, dry eye disease, ocular irritation or a combination of these events (WEINREB, R. N., AUNG, T., MEDEIROS, F. A. The pathophysiology and treatment of glaucoma: a review. JAMA, (18):1901-11, 2014). Moreover, patients would be highly benefited by drugs acting on the conventional outflow (i.e. improving outflow through the TM, SC and distal scleral vessels). As the conventional outflow is the main outflow, the drugs that act by this pathway tend potential to be more potent than the drugs that act thought unconventional outflow, or at least could have a complementary action. Moreover, a drug that could act causing blood flow increase to the optic nerve could directly prevent or delay the optic nerve damage (i.e. neuroprotection). The convenient topical administration, preferably with a low frequency of treatment, is also a valuable nice-to-have feature. Considerable efforts have been made in this direction, but with very limited success (WEINREB, R. N., AUNG, T., MEDEIROS, F. A. The pathophysiology and treatment of glaucoma: a review. JAMA, (18):1901-11, 2014; BRAUNGER, B. M., FUCHSHOFER, R., TAMM, E. R. The aqueous humor outflow pathways in glaucoma: A unifying concept of disease mechanisms and causative treatment. Eur J Pharm Biopharm, 95(Pt B):173-81, 2015).

### Role of NO in Glaucoma

Nitric Oxide (NO) has gained a lot of attention recently as a potential new target for the treatment of glaucoma. The biologic effects of NO could simultaneously mediate increased AH drainage through the conventional outflow and protect the optical nerve from further injury (CAVET, M. E., VITTITOW, J. L., IMPAGNATIELLO, F., ONGINI, E., BASTIA, E. Nitric oxide (NO): an emerging target for the treatment of glaucoma. Invest Ophthalmol Vis Sci, 55(8): 5005-5015, 2014; ALIANCY, J., STAMER, W. D., WIROSTKO, B. A Review of Nitric Oxide for the Treatment of Glaucomatous Disease. Ophthalmol Ther, 6(2): 221-232, 2017; WAREHAM, L. K., BUYS, E. S., SAPPINGTON, R. M. The nitric oxide-guanylate cyclase pathway and glaucoma. Nitric Oxide, 77: 75-87, 2018).

Recent evidence indicates that the NO signaling pathway has a role in the ocular homeostasis, regulating AH drainage and, therefore, IOP. In healthy human eyes, the capacity to form NO is found in the anterior ocular tissues. Precisely, the neuronal nitric oxide synthase (nNOS) isoform is expressed in the ciliary non-pigmented epithelium, astrocytes of the optic nerve head, and in the lamina cribrosa; endothelial NOS (eNOS) is found in the ciliary muscle, TM, and SC as well as in the retinal vasculature; inducible NOS (iNOS) is not constitutively expressed in the eye at physiological conditions, only expressed only after stimulation in macrophages located in the stroma and in the ciliary process, and in astrocytes (WAREHAM, L. K., BUYS, E. S., SAPPINGTON, R. M. The nitric oxide-guanylate cyclase pathway and glaucoma. Nitric Oxide, 77: 75-87, 2018).

The ocular anatomic structures that regulate the AH drainage are formed by contractile tissues. For instance, TM cells are known to be highly contractile in nature, analogous to vascular smooth muscle cells (VSMC), in which the role of NO-cGMP signaling in endothelium-dependent relaxation is well understood (CAVET, M. E., VITTITOW, J. L., IMPAGNATIELLO, F., ONGINI, E., BASTIA, E. Nitric oxide (NO): an emerging target for the treatment of glaucoma. Invest Ophthalmol Vis Sci, 55(8): 5005-5015, 2014). Studies using isolated segments of the TM in vitro showed that L-NAME, an unspecific NOS antagonist, decreases the flow rate, thus supporting the role of NO, which in the TM is generated by iNOS (SCHNEEMANN, A., DIJKSTRA, B. G., VAN DEN BERG, T. J., KAMPHUIS, W., HOYNG, P. F. Nitric oxide/guanylate cyclase pathways and flow in anterior segment perfusion. Graefes Arch Clin Exp Ophthalmol. 240(11): 936-41, 2000; ALIANCY, J., STAMER, W. D., WIROSTKO, B. A Review of Nitric Oxide for the Treatment of Glaucomatous Disease. Ophthalmol Ther, 6(2): 221-232, 2017).

The SC consists of endothelial cells and connective tissue, similar in structure to a vein. Contractility of these cells plays a role in the modulation of aqueous outflow and therefore these cells are a potential site of action for NO (CAVET, M. E., VITTITOW, J. L., IMPAGNATIELLO, F., ONGINI, E., BASTIA, E. Nitric oxide (NO): an emerging target for the treatment of glaucoma. Invest Ophthalmol Vis Sci, 55(8): 5005-5015, 2014).

In vitro studies in human SC cells, demonstrate that inhibition of endogenous NOS with L-NAME results in an increase in cell volume, suggesting that in vivo reduction in NO levels may increase outflow resistance, and thereby elevate IOP. These findings are in line with in vivo data, which show that transgenic mice overexpressing eNOS in vascular endothelium including SC have reduced IOP and increased outflow facility compared with wild-type mice (CAVET, M. E., VITTITOW, J. L., IMPAGNATIELLO, F., ONGINI, E., BASTIA, E. Nitric oxide (NO): an emerging target for the treatment of glaucoma. Invest Ophthalmol Vis Sci, 55(8): 5005-5015, 2014).

In patients with POAG, the abundance of eNOS was decreased in the TM, SC, and ciliary muscle, suggesting that reduced NO production might contribute to IOP elevation. In addition, NO levels were decreased in the AH of patients with POAG (CAVET, M. E., VITTITOW, J. L., IMPAGNATIELLO, F., ONGINI, E., BASTIA, E. Nitric oxide (NO): an emerging target for the treatment of glaucoma. Invest Ophthalmol Vis Sci, 55(8): 5005-5015, 2014).

The optic nerve head—the site of glaucomatous axonal injury—is supplied by the posterior ciliary artery circulation and retinal circulation. Posterior ciliary artery, the main source of blood supply, branching off the ophthalmic artery, divides later into a number of short posterior ciliary arteries that enter the globe around the optic nerve, and contribute to the perfusion of the anterior optic nerve head. The surface nerve fiber layer of the retina is fed by arteriolar branches from the central retinal artery. In the retina and optic nerve head, endogenous NO is essential for maintaining basal blood flow. (SAMPLES, J. R., KNEPPER, P. A. Glaucoma Research and Clinical advances: 2018 to 2020. Amsterdam, The Netherlands: Kugler Publications, New concepts in glaucoma, v.2, 2018). POAG and NTG have both been associated with peripheral vascular endothelial dysfunction, presenting decreased NO bioavailability and local alterations of the NO signaling system (GIACONI, J. A., LAW, S. K., COLEMAN, A. L., CAPRIOLI, J. Pearls of Glaucoma management, Springer, 2010). Animal studies confirmed that NO-induced IOP lowering is mediated predominantly via an increase in conventional outflow facility and the therapeutic potential of NO has been recently validated in patients with POAG. Nitrovasodilators could be considered as a new class of ocular hypotensive agents, considering that NO mediates a multitude of diverse ocular effects and maintenance of IOP. NO donors have been shown to mediate IOP-lowering effects in both preclinical models and clinical studies, primarily through cell volume and contractility changes in the conventional outflow tissues. A NO-donating associated with a prostaglandin F receptor agonist, latanoprostene bunod, was more effective than the reference compound, latanoprost, in lowering IOP. The dual-mode of action combining Prostaglandin F receptor activation and NO donation increases AH outflow through both unconventional and conventional pathways simultaneously (CAVET, M. E., VITTITOW, J. L., IMPAGNATIELLO, F., ONGINI, E., BASTIA, E. Nitric oxide (NO): an emerging target for the treatment of glaucoma. Invest Ophthalmol Vis Sci, 55(8): 5005-5015, 2014).

Because of NO's vasodilation effect and its likely role in optic nerve head blood flow regulation, a NO-based therapy that enhances optic nerve and retinal vascular NO signaling may have the potential to exert beneficial effects on injured RGC (TSAI, J. C., GRAY, M. J., CAVALLERANO, T. Nitric oxide in glaucoma: what clinicians needs to know. Candeo Clinical/Science Communications, LLC, 2017). However, no current therapy was able to deliver NO to the retina in a sufficient concentration to provoke vasodilatation in the arteries that supply the optic nerve head and therefore protected it from ischemic injuries secondary to glaucoma and other ischemic optic neuropathy, as non-arteritic ischemic optic neuropathy (NAION).

### Peptides Targeting Eye Diseases

Therapeutic peptides have shown great promise as novel therapeutics in the treatment of ocular diseases. These molecules offer several advantages such as high potency, low unspecific binding, less toxicity, and minimization of drug-drug interaction. However, factors such as physical and chemical degradation, short in vivo half-lives, clearance by the mononuclear phagocytes (MPS) of the reticulum endothelial system (RES), risk of immunogenicity, and failure to permeate cell membranes pose high challenges to topic ocular administration of peptides. These barriers have warranted large efforts to allow the effective use of therapeutic peptides in the treatment of eye diseases (MANDAL, A., PAL, D., AGRAHARI, V., TRINH, H. M., JOSEPH, M., MITRA, A. K. Ocular delivery of proteins and peptides: Challenges and novel formulation approaches. Adv Drug Deliv Rev, 126: 67-95, 2018).

There are currently five biologic drugs (three monoclonal antibodies, one aptamer, and one therapeutic protein) approved for the treatment of eye diseases. However, none of them targets glaucoma. Moreover, those drugs are administered either subcutaneously or intraocularly. The repeated eye injections correlate with an increased propensity for complications, such as endophthalmitis, cataracts, retinal tears, and retinal detachment, thus representing a significant inconvenience for patients.

U.S. Pat. No. 9,279,004 discloses a peptide with 19 amino acids (PnTx(19)) and molecular weight of 2,485.85 Da, built from the toxin PnTx2-6. The natural toxin causes priapism in male patients bitten by the spider Phoneutria nigriventer. In turn, the peptide PnTx(19), also called PnPP-19, is a non-naturally occurring molecule, engineered from non-contiguous domains of the natural toxin. The disclosures reveal that PnPP-19 is capable of enhancing the erectile function, as demonstrated by an improved relaxation of isolated strips of murine penile corpus cavernosum ex vivo.

Further research demonstrated that PnPP-19-induced relaxation is mediated by the activation of NOS enzymes, production of NO, downstream activation of sGC, and cGMP signaling (SILVA, C. N., NUNES, K. P., TORRES, F. S., CASSOLI, J. S., SANTOS, D. M., ALMEIDA, Fde. M., MATAVEL, A., CRUZ, J. S., SANTOS-MIRANDA, A., NUNES, A.D., CASTRO, C. H., MACHADO DE ÁVILA, R. A., CHÁVEZ-OLÓRTEGUI, C., LÁUAR, S. S., FELICORI, L., RESENDE, J. M., CAMARGOS, E. R., BORGES, M. H., CORDEIRO, M. N., PEIGNEUR, S., TYTGAT, J., DE LIMA, M. E. PnPP19, a synthetic and nontoxic peptide designed from a Phoneutria nigriventer Toxin, potentiates erectile function via NO/cGMP. J Urol; 194(5): 1481-90. 2015). Thus, PnPP-19 was claimed as a potential candidate for the treatment of erectile dysfunction, with a potential application in patients that are resistant to the therapy based on phosphodiesterase 5 inhibitors (PDE5i).

Novel and unpublished results show that PnPP-19 is able to permeate the eye and reduce IOP in animals with healthy eyes. Due to its proprieties to permeate in the eye and reach the retina, further investigations have demonstrated that PnPP-19 also has neuroprotective properties, protecting the retina and optic nerve from the damage caused by retinal ischemia in animal models of optic nerve damage. Therefore, the present approach departs from this discovery to derive pharmaceutical compositions and methods of treating and/or preventing eye diseases related to intraocular hypertension and/or optic nerve degeneration, such as PACG, POAG, NTG, age-related macular degeneration and diabetes retinopathy.

### SUMMARY

The present specification describes methods of treatment and pharmaceutical compositions comprising a NOS-enhancer peptide that can simultaneously improve the conventional outflow of AH and directly prevent the progression of optic nerve degeneration. Therefore, the methods and compositions described herein are useful for treating and/or preventing eye diseases related to intraocular hypertension and/or optic nerve degeneration, such as PCAG, POAG, NTG, and elevated IOP.

The present invention unexpectedly shows that when topically administered as eye drops, the synthetic peptide PnPP-19 is capable of penetrating the eye, reducing the IOP in animals with healthy eyes and also glaucomatous untreated eyes, and protecting against ischemic injury to the retina and optic nerve. Accordingly, the embodiments of this description include the following:

1—A method of reducing the intraocular pressure, comprising topically administering to an eye an effective amount of PnPP-19.
2—A method of treating or preventing ischemic optic neuropathy, comprising topically administering to an eye an effective amount of PnPP-19.
3—The method of ##1 or 2 wherein the administration is one or two drops per day of a composition comprising, in a pharmaceutically acceptable liquid medium, PnPP-19 in an effective amount, specifically between 0.08 to 0.72% of peptide per volume.
4—The method of #2 wherein the ischemic optic neuropathy is glaucoma.
5—The method of #4 wherein the glaucoma is normal-tension glaucoma.
6—The method of #2 wherein the ischemic optic neuropathy is age-related macular degeneration, diabetic neuropathy or non-arteritic ischemic optic neuropathy (NAION).
7—The method of ##1 or 2 wherein administering PnPP-19 starts before vision loss of the eye.

8—The method of ##1 or 2 wherein administering PnPP-19 starts before partial vision loss of the eye due to intraocular pressure or ischemic optic neuropathy.

9—The method of ##8, wherein the administering PnPP-19 continues after partial vision loss of the eye due to intraocular pressure or ischemic optic neuropathy.
10—The method of ##1 or 2 wherein administering PnPP-19 starts after partial vision loss caused by ischemic optic neuropathy of the eye.
11—A pharmaceutical composition for ophthalmic administration, comprising, in a pharmaceutically effective medium, an effective amount of PnPP-19, specifically between 0.08 to 0.72% of peptide per volume, and one or more pharmaceutically acceptable excipients.
12—A METHOD OF TREATING GLAUCOMA IN A PATIENT, comprising topically administering to an eye of a patient in need thereof, a composition formulated for ophthalmic administration comprising, in a pharmaceutically acceptable medium, an effective amount of PnPP-19, specifically between 0.08 to 0.72% of peptide per volume.
13—The method of #12, wherein the patient has normal intraocular tension.
14—A METHOD OF TREATING A PATIENT WITH OCULAR HYPERTENSION, comprising topically administering to an eye of a patient in need thereof, a composition formulated for ophthalmic administration comprising, in a pharmaceutically acceptable medium, an effective amount of PnPP-19, specifically between 0.08 to 0.72% of peptide per volume.
15—A method for the REDUCTION OF INTRAOCULAR PRESSURE IN A PATIENT, comprising topically administering to an eye of a patient in need thereof, an effective amount of PnPP-19, specifically between 0.08 to 0.72% of peptide per volume, formulated for ophthalmic administration comprising, in a pharmaceutically acceptable medium.
16—The method of ##14 or 15, wherein the patient has elevated intraocular pressure.
17—The method of #16, wherein the patient has glaucoma.

In some embodiments, practicing ##1-17 make it possible to achieve one or more of the following:

PnPP-19 applied topically (one eye drop, with 80 μg of peptide in 20 μL saline (0.4%)) permeates from the cornea, thought vitreous body to the retinal epithelium.
PnPP-19 applied topically (one eye drop, with 80 μg of peptide in 20 μL saline (0.4%)) increase the NO levels inside the eye, confirmed in animal model with nitrite quantification in comparison to placebo.
PnPP-19 applied topically (one eye drop, with 80 μg of peptide in 20 μL saline (0.4%)) decreases significantly IOP up to 24 hours, both in normotensive as in glaucoma model rat, without causing ocular irritation, or corneal nor retinal damage.
PnPP-19 also has neuroprotective effects, as vision preservation, reduction of histological damage, protection of retinal cells against ischemic injury, in either prevention or therapeutic mode of treatment, confirmed in an animal model of retinal ischemia.
PnPP-19 applied topically (one eye drop, with 250 μg of peptide in 50 μL saline (0.5%)) is safe and tolerable, and capable of decrease the IOP in healthy human subjects.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1—HET-CAM's Photographs after 5 minutes exposure to (A) 0.1M NaOH (positive control); (B) NaCl 0.9% (negative control); (C-F) PnPP-19, at concentrations that vary from 40 μg of peptide in 20 μL (0.2%) to 320 μg of peptide in 20 μL (1.6%).

FIG. 2—PnPP-19 does not affect retinal vessels. Photograph of the indirect fundus, representing ophthalmoscopy of the rat's retina, before and one, seven and fifteen days after treatment with 40-160 μg of peptide in 20 μL of saline (0.2 to 0.8%).

FIG. 3—PnPP-19 does not alter the retinal morphology. Sequence of illustrative photographs of histological layers of the retina 15 days after PnPP-19 instillation, showing in PnPP-19 40 μg (0.2%); PnPP-19 80 μg (0.4%); PnPP-19 160 μg (0.8%); and Control, n=4. RPE-Retinal Pigment Epithelium, ONL-Outer nuclear layer, INL-Inner nuclear layer, GCL-Ganglion cell layer. Digital images were obtained using a microscope (Apotome.2, ZEISS, Germany) with a 20× objective.

FIG. 4—PnPP-19 does not alter the cornea morphology. Sequence of illustrative photographs of histological layers of the cornea 15 days after PnPP-19 instillation, showing in PnPP-19 40 μg (0.2%); PnPP-19 80 μg (0.4%); PnPP-19 160 μg (0.8%); and Control, n=4. Epithelium layer, Stroma layer, Endothelium layer. Digital images were obtained using a microscope (Apotome.2, ZEISS, Germany) with a 20× objective.

FIG. 5—PnPP-19 reduces IOP in normotensive rats. Comparison between PnPP-19 (80 μg/eye, 0.4%) and control results. The bars represent the % Δ IOP decrease of PnPP-19 after subtraction of control effect. n=8.

FIG. 6—PnPP-19 reduces IOP in rats with glaucoma. Comparison between healthy rats and treated and untreated animals with PnPP-19 (80 μg/eye, 0.4%). Result expressed in mmHg. Asterisks represent statistical difference in relation to the untreated: *p<0.5; **p<0.01; ***p<0.001, two way ANOVA with Bonferroni post-test.

FIG. 7—PnPP-19 preserves the number of RGCs. There was a smaller number of RGCs in the retinas of glaucomatous animals compared to healthy rats. Glaucomatous animals treated with PnPP-19 (80 μg/eye, 0.4%) have an RGC count higher than untreated glaucomatous rats and was not statistically different compared to healthy rat. Asterisks represent statistical difference in relation to the healthy animals: **p<0.01, one way ANOVA.

FIG. 8—PnPP-19 permeates the cornea and reaches the retina. Comparison between Control (saline) and the treatment group using PnPP-19 (80 μg/eye, 0.4%). The images represent the fluorescence intensity (color green) from the cornea (A), vitreous body (B) and retina (C). The charts on the right represent the fluorescence intensity from the cornea and retina. Eyes were removed 3 hours after application of one drop (20 μl). Fluorescence microscopy performed using APOTOME.2 ZEISS, 10× objective, the bar is 100 μm in length. FITC was excited at 490 nm and the emission was detected at 526 nm. Asterisks represent statistical difference in relation to control: ***p<0.001, Student's t test

FIG. 9—PnPP-19 reduces the histological damage caused by retinal ischemia. Effects of instillation of PnPP-19 (80 μg/eye, 0.4%) after ischemia induction. Retinal sections were stained with hematoxylin-eosin. Bars=50 μm. Black arrows denote areas of vacuolization and pyknotic nuclei. Red arrows denote an increase in OS and RPE layers. (A) Ischemic/untreated; (B) ischemic/PnPP-19 post-treatment; (C) healthy. RPE-Retinal Pigment Epithelium, OS-outer segment, ONL-outer nuclear layer, INL-inner nuclear layer, GCL-ganglion cell layer.

FIG. 10—PnPP-19 reduces vision loss. Effects of instillation of PnPP-19 (80 μg/eye, 0.4%) after ischemia induction on ERG curves. Comparison between Ischemic/untreated and ischemic/PnPP-19 post-treatment regarding b-amp/a-amp ratio in ischemic retinas. n=6.

FIG. 11—PnPP-19 avoids the histological damage caused by retinal ischemia. Effects of instillation of PnPP-19 (80 μg/eye, 0.4%) before ischemia induction. Retinal sections were stained with hematoxylin-eosin. Bars=50 μm. Black arrows denote areas of vacuolization and pyknotic nuclei. (A) ischemic/untreated; (B) ischemic/PnPP-19 pre-treatment; (C) healthy. RPE-Retinal Pigment Epithelium, OS-outer segment, ONL-outer nuclear layer, INL-inner nuclear layer, GCL-ganglion cell layer.

FIG. 12—PnPP-19 prevents vision loss. Effects of instillation of PnPP-19 (80 μg/eye, 0.4%) before ischemia induction on ERG curves. Comparison between Ischemic/untreated and ischemic/PnPP-19 pre-treatment regarding b-amp/a-amp ratio in ischemic retinas. n=6.

FIG. 13—PnPP-19 increases nitrite levels in the eye. The tissues of the normotensive eyes were collected 2 hours after local instillation of vehicle (saline) or PnPP-19 (80 μg/eye, 0.4%). Each column represents the mean +/−SEM. n=6. ***p<0.0001, the Student T test for nonpaired data.

FIG. 14—PnPP-19 decreases the IOP in humans. IOP was measured by non-contact tonometer before instillation (basal) and after 6 h of instillation. n=12.

### DESCRIPTION OF EMBODIMENTS

The method of treatment of the present comprises the administration of PnPP-19 to a patient in need thereof. As used henceforth, the name PnPP-19 relates to the polypeptide having a sequence of SEQ ID NO 1: Gly Glu Arg Arg Gln Tyr Phe Trp Ile Ala Trp Tyr Lys Leu Ala Asn Ser Lys Lys, which is optionally N-terminal acetylated and/or C-terminal amidated.

### Definitions

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise.

It is further to be understood that all base sizes or amino acid sizes, and all molecular weight or molecular mass values, given for polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety.

Peptide or polypeptide is a polymer in which the monomers are amino acid residues which are joined together through amide bonds. When the amino acids are alpha-amino acids, either the L-optical isomer or the D-optical isomer can be used, the L-isomers being preferred. The terms “polypeptide,” “peptide,” or “protein” as used herein are intended to encompass any amino acid sequence and include modified sequences such as glycoproteins. The terms “polypeptide” and “peptide” are specifically intended to cover naturally occurring proteins, as well as those which are recombinantly or synthetically produced. The abbreviations for amino acid residues are the standard code of 3 letters and/or 1 letter used in the art for reference to one of the common 20-L amino acids.

The term “therapeutic activity” as used herein refers to a demonstrated or potential biological activity whose effect is consistent with a desirable therapeutic outcome in humans, or to desired effects in non-human mammals or in other species or organisms. A given therapeutic peptide may have one or more therapeutic activities, however, the term “therapeutic activities’ as used herein may refer to a single therapeutic activity or multiple therapeutic activities. “Therapeutic activity” includes the ability to induce the desired response and may be measured in vivo or in vitro. For example, a desirable effect may be assayed in cell culture, isolated tissues, animal models, clinical evaluation, EC₅₀ assays, IC₅₀ assays, or dose-response curves. The term therapeutic activity includes preventive or curative treatment of a disease, disorder, or condition. Treatment of a disease, disorder or condition can include improvement of a disease, disorder or condition by any amount, including the elimination of a disease, disorder or condition.

The term “therapeutically effective” as used herein depends on the condition of a subject and the specific compound administered. The term refers to an amount effective to achieve a desired clinical effect. A therapeutically effective amount varies with the nature of the condition being treated, the length of time that activity is desired, and the age and the condition of the subject, and ultimately is determined by the health care provider. In one aspect, a therapeutically effective amount of a peptide or composition is an amount effective enough to reduce at a clinically significant level the IOP of an individual in need thereof, thus inhibiting, reducing or preventing optic neuropathies associated with retinal ganglion cell death, retinal nerve fiber layer thinning and optic nerve head cupping, such as glaucoma.

As used herein the term “no observable effect level” (NOEL) denotes the highest dose tested in an animal species with no detected effects. The term “no observable adverse effect level” (NOAEL) denotes the highest dose level that does not produce a significant increase in adverse effects in comparison to the control group. The term “maximum tolerated dose” (MTD) denotes the highest dose that does not produce unacceptable toxicity in a toxicity study. The term “human equivalent dose” (HED) denotes a dose in humans anticipated providing the same degree of effect as that observed in animals at a given dose.

As used herein, “conservative amino acids substitutions” are substitutions that do not result in significant modification of the IOP-reducing activity or tertiary structure of a given polypeptide or protein. Such substitutions typically involve the substitution of an amino acid residue selected by a different residue having similar physicochemical properties. For example, the substitution of Glu with Asp is considered a conservative substitution, since they are both negatively charged amino acids of similar size. Grouping of amino acids by their physicochemical properties is known to those skilled in the art.

The “similarity” between two sequences is determined by comparing the sequences of amino acids of the polypeptides when aligned in order to maximize the superposition, minimizing the gaps of the sequence, followed by an accounting of identical residues between the sequences. The percentage of identity of two sequences of amino acids or nucleic acids can be determined by visual inspection and/or mathematical calculation, commonly done for longer sequences comparing the information of the sequence using a computer program. Examples of programs that can be used by a person skilled in the art for comparison of sequences of peptides and nucleic acids are the BLAST (BLASTP) and BLASTN, freely available on the website of the National Library of Medicine http://www.ncbi.nlm.nih.gov/BLAST. In preferred modalities, the sequences are considered homologous or identical to one another if their amino acid sequences are at least 50% identical, more preferably if the sequences are 70% or 75% identical, still more preferably if the sequences are 80% or 85% identical, still more preferably if the sequences are 90 or 95% identical, when determined from a visual inspection or an adequate computer program.

A peptide fragment is “derived from” an original peptide if there is a sequence of amino acids that is identical or homologous to the sequence of amino acids of the original peptide or polypeptide. The said fragment may be produced by synthetic methods (e.g. solid-state peptide synthesis, recombinant DNA expression in a modified cell and enzymatic degradation in vitro) or natural degradation of an original peptide. In the latter case, the said fragment results from a process occurred in a living organism (i.e. an isolated cell or a tissue in vitro, or an animal, for instance, but not limited to, a human being, in vivo), therefore, a product of the metabolism of the said organism. The said product or products from the metabolic degradation of an original peptide (or drugs in general) is called metabolite. Such metabolites may or may not have a biological effect. When such metabolites still have a biological activity that resembles the original peptide, they are considered active metabolites. Therefore, those skilled in the art could promptly capture that fragments of a therapeutic peptide, produced either synthetically or naturally, may exhibit lower, equal or higher IOP-lowering activity than the original peptide.

“Ocular neuropathies” or “neuropathic diseases” as mentioned herein are defined as the acute or progressive degeneration of the nervous tissues of the eyes (e.g. the retina and the optic nerve). Such ocular neuropathies may or may not be related to intraocular hypertension and include, but are not limited to, POAG, PACG, NTG, age-related macular degeneration, diabetic retinopathy and NAION.

“Elevated pressure”, or “ocular hypertension” is defined as IOP two standard deviations above the mean IOP. (16 mmHg) (BOEY, P. Y., MANSBERGER, S. L. Ocular hypertension: an approach to assessment and management. Can. J. Ophthalmol. 49(6):489-96, 2014). Therefore, elevated pressure can be considered if measured IOP is higher than 21 mmHg. IOP is typically measured by applanation tonometry, which gives an estimate of the pressure inside the eye based on the resistance to flattening of a small area of the cornea. Variances in diurnal IOP are normal, with higher values found in the morning.

### Method of Treatment

A composition comprises, in a pharmaceutically acceptable medium, an effective amount of a polypeptide having a sequence of SEQ ID NO 1, which is optionally N-terminally acetylated and/or C-terminally amidated. The polypeptide is referred to herein as PnPP-19. In some embodiments, the polypeptide is in the form of a pharmaceutically acceptable salt. In some embodiments, the polypeptide is acetylated, e.g. in some embodiments, using acetyl group into the N-terminus (peptide glycine (G)). In some embodiments, the polypeptide is amidated, e.g. in some embodiments, using amide group into the C-terminus (peptide lysine (K)). In some embodiments, the polypeptide is acetylated and amidated.

A method comprises, administering an effective amount of PnPP-19 a pharmaceutical composition noted herein. In some embodiments, the method is for reducing the IOP. In some embodiments, the method if for treating or preventing ischemic optic neuropathy, such as glaucoma, including normal-tension glaucoma; age-related macular degeneration; or diabetic neuropathy.

In some embodiments, the patient is a human. In some embodiments, the patient him/herself administers. In other embodiments, a healthcare professional administers to the patient.

In some embodiments, the administering is to the patient's affect eye. In other embodiments, administering is to the patient's unaffected eye. In some embodiments, administering is to both the patient's eyes (affected or unaffected or combinations thereof).

In some embodiments, administering starts before vision loss of the eye. In some embodiments, administering starts before partial vision loss of the eye due to ocular hypertension or ischemic optic neuropathy.

In some embodiments, the administering PnPP-19 starts before partial vision loss of the eye. In some embodiments, the administering PnPP-19 starts after partial vision loss of the eye. In some embodiments, the administering PnPP-19 starts before and continues after partial vision loss of the eye.

Administering, in some embodiments, is topical to the patient's eye. In some embodiments, the administering is in the form of drops. In other embodiments, the administering is in the form of sprays.

The preferred embodiment of this description relates to methods of treating and/or preventing eye diseases related to ocular hypertension and/or optic nerve degeneration, such as glaucoma, based on the administration of a NOS-enhancer peptide to a person in need thereof.

### Diagnosis of IOP/Glaucoma

Increased IOP and glaucoma are usually asymptomatic early in the disease course and patients are usually identified through screening or based on abnormal findings on an eye examination. Due to the lack of initial symptoms or signs, more than 50% of glaucoma cases go undiagnosed (TOPOUZIS, F., COLEMAN, A. L., HARRIS, A. Factors associated with undiagnosed open-angle glaucoma: the Thessaloniki Eye Study. Am J Ophthalmol, 145: 327-335, 2008).

The screening in asymptomatic patients is more useful and cost-effective if targeted at the population at high risk for glaucoma, such as older adults, persons with a family history of glaucoma and African American and Hispanic Population.

Half of all POAG patients have a positive family history of the disease (AWADALLA, M. S., FINGERT, J. H., ROOS, B. E. Copy number variations of TBK1 in Australian patients with primary open-angle glaucoma. Am J Ophthamol, 159: 124-130, 2015), and at least one study showing that 60% of glaucoma patients were found to belong to families in which others were afflicted with the disease (GREEN, M. G., KEARNS, L. S., WU, J. How significant is a family history of glaucoma. Experience from the Glaucoma Inheritance Study in Tasmania. Clin Exp Ophthalmol, 35: 793-799, 2007). Myopia is a significant risk factor for glaucoma, especially in those of Asian descent (MCMONNIES, C. W. Glaucoma history and risk factors. J Optom, 10(2): 71-78, 2017). There is evidence for myocilin mutations in advanced POAG and of copy number variations of TBK1 in NTG illustrating the contribution of genetics for glaucoma risk prediction (SOUZEAU, E., BURDON, K. P., DUBOWSKY, A. Higher prevalence of myocilin mutations in advanced glaucoma in comparison with less advanced disease in an Australian Disease Registry. Ophthalmology, 120(6): 1135-1143, 2013; AWADALLA, M. S., FINGERT, J. H., ROOS, B. E. Copy number variations of TBK1 in Australian patients with primary open-angle glaucoma. Am J Ophthamol, 159:124-130, 2015). Women are at higher risk of PACG, and there is no gender predilection for POAG (VAJARANANT, T. S., NAYAK, S., WILENSKY, J. T., JOSLIN, C. E. Gender and glaucoma: what we know and what we don't know. Curr Opin Ophthalmol, 21: 91-99, 2010).

A patient who has progressed to definite glaucoma may have sufficient visual field loss to complain of impaired night driving, near vision, reading speed, or outdoor mobility.

In high-risk subpopulations or symptomatic population, an eye examination will diagnosis glaucoma if one of the following conditions are present: (i) consistently elevated IOP, (ii) suspicious-appearing optic nerve (such as abnormal nerve fiber layer on optical coherence tomography (OCT) or disc hemorrhage), or (iii) abnormal visual field. (STANLEY, J., HUISINGH, C. E., SWAIN, T. A., MCGWIN, G. JR., OWSLEY, C., GIRKIN, C. A., RHODES, L. A. Compliance With Primary Open-angle Glaucoma and Primary Open-angle Glaucoma Suspect Preferred Practice Patterns in a Retail-based Eye Clinic. J Glaucoma, 27(12):1068-1072, 2018).

### PnPP-19 for the Treatment of Glaucoma

Since the discovery of the role of NO as a critical endogenous mediator in 1987, research on this molecule has rapidly flourished and expanded in many directions, especially in those diseases characterized by perturbations in NO production/signaling.

NO is generated endogenously by a family of enzymes (NOS) and, in the eye, it is important for modulating the dynamic balance between the rate of secretion (inflow) and drainage (outflow), and thus IOP regulation. In the healthy eye, eNOS activity guarantees a NO supply in the AH outflow pathway and ciliary muscles, maintaining an adequate balance between inflow and outflow. In glaucomatous eyes, however, endothelial dysfunction leads to eNOS activity is decreased in the TM, SC and ciliary muscles, resulting in low NO levels in those areas, leading to a misbalance between AH inflow and outflow and in IOP increase. (CAVET, M. E., VITTITOW, J. L., IMPAGNATIELLO, F., ONGINI, E., BASTIA, E. Nitric oxide (NO): an emerging target for the treatment of glaucoma. Invest Ophthalmol Vis Sci, 55(8): 5005-5015, 2014)

The administration of NO donors (eg, nitroglycerin, sodium nitroprusside) leads to IOP lowering in several animal models as well as in humans. NO induction increases the outflow facility through the relaxation of the TM and the inner wall of SC, called conventional outflow, but also leads to relaxation of the ciliary muscles, altering the uveoscleral outflow pathway (also called non-conventional outflow pathway) (CAVET, M. E., VITTITOW, J. L., IMPAGNATIELLO, F., ONGINI, E., BASTIA, E. Nitric oxide (NO): an emerging target for the treatment of glaucoma. Invest Ophthalmol Vis Sci, 55(8): 5005-5015, 2014). However the NO donors have difficulties to deliver their payloads effectively and are not targeted, leading to low NO offer in the target tissue, or high NO deliver, provoking systemic side effects and nitrosylation of the cornea, iris, and TM).

PnPP-19 enhances the production of iNOS and nNOS. The nNOS is expressed in the ciliary non-pigmented epithelium, and the iNOS is expressed in almost all cells, including the ciliary body (uveoscleral, non-conventional pathway) and TM and SC (conventional outflow). Therefore, even in the physiopathological condition of endothelial dysfunction and low eNOS activity, PnPP-19 will still be able to increase NO levels due to increased activity of iNOS and nNOS.

PnPP-19 demonstrated significantly IOP lowering capacity in animal models. iNOS can produce a large quantity of NO (100 to 1000 times greater compared to eNOS) and for a prolonged period of time (cellular half-life is 3 h). PnPP-19, as an iNOS enhancer, is capable to lower IOP up to 24 h with one daily administration, and the reduction of IOP is sustained during this period, without great variation in IOP, a desirable effect to avoid vision loss. Moreover, due to PnPP-19 mechanism of action, NO is produced locally in the targeted cells and therefore avoiding lack of effect due to low NO levels or side effects due to NO action outside the target. PnPP-19 demonstrates to be non-irritant and to not cause corneal or retinal damage in preclinical studies.

Glaucomatous eyes, like POAG and NTG, have peripheral vascular endothelial dysfunction, low eNOS activity and decreased NO levels, leading to ischemic damage in the optic nerve head. The nNOS and iNOS are also expressed in astrocytes of the optic nerve head. PnPP-19 is capable to permeate the cornea and reach the retina, and therefore to act enhancing nNOS and iNOS in the optic nerve head, in the arteries that supply this region and also in the astrocytes and nerves. The vasodilatation effect of NO produced by the increased activity can restore the ischemia in the head optic nerve and avoid the damage and cell death. The neuroprotective effect of PnPP-19 was confirmed both when administered before the ischemic induction (prophylaxis) as after ischemic induction (treatment). PnPP-19 is capable of protecting the retina against ischemic injury by reduction of histological damage, preservation of RGCs, and avoiding or reducing vision loss.

### PnPP-19 in the Treatment of Age-Related Macular Degeneration (AMD)

Age-related macular degeneration is the leading cause of visual loss and blindness among persons 60 years or older in developed countries (FRIEDMAN, D. S., O'COLMAIN, B. J., MUÑOZ, B., TOMANY, S. C., MCCARTY, C., DE JONG, P. T., NEMESURE, B., MITCHELL, P., KEMPEN, J.; EYE DISEASES PREVALENCE RESEARCH GROUP. The Eye Diseases Prevalence Research Group. Prevalence of age-related macular degeneration in the United States. Arch Ophthalmol. 2004; 122:564-572).

Patients with AMD are classified as early stage when the visual function is affected; intermediate stage, with progressive worsening of the symptoms; and late-stage when central vision is severely compromised or entirely lost. The pathogenesis of early AMD is characterized by a thickening if Bruch Membrane, due to lipid and protein accumulation, that leads to the formation of sub-retinal pigment epithelial cells (RPE) deposits that occur as discrete accumulations called drusen, hallmark lesions of AMD. Late-stage AMD can present in two forms: a “dry”, atrophic form of AMD, characterized by macular drop out of RPE and photoreceptors, termed geographic atrophy, and “wet” neovascular form of AMD, characterized by the invasion of RPE and/or the retina by abnormal blood vessels, therefore neovascular, or exudative AMD, since this presentation involves choroid neovascularization (RICKMAN, C. B., FARSIU, S., TOTH, C. A., KLINGEBORN, M. Dry Age-Related Macular Degeneration: Mechanisms, Therapeutic Targets, and Imaging. Invest Ophthalmol Vis Sci, 54(14): ORSF68-ORSF80, 2013).

The principal risk factors for developing AMD are age, cataract surgery, medical history of hypertension; and for late AMD, smoking (ANASTASOPOULOS, E., HAIDICH, A. B., COLEMAN, A. L., WILSON, M. R., HARRIS, A., YU, F., KOSKOSAS, A., PAPPAS, T., KESKINI, C., KALOUDA, P., KARKAMANIS, G., TOPOUZIS, F. Risk factors for Age-related Macular Degeneration in a Greek population: The Thessaloniki Eye Study. Ophthalmic Epidemiol, 25(5-6): 457-469, 2018).

It is known that choroidal blood flow is regulated by NO generated by both eNOS present in endothelial cells, and nNOS present in perivascular nitrergic neurons, with nNOS being the main source of NO in arterioles (GRIFFITH, O. W., STUEHR, D. J. Nitric oxide synthases: properties and catalytic mechanism. Annu Rev Physiol, 57: 707-36, 1995; KASHIWAGI, S., KAJIMURA, M., YOSHIMURA, Y., SUEMATSU, M. Nonendothelial source of nitric oxide in arterioles but not in venules: alternative source revealed in vivo by diaminofluorescein micro fluorography. Circ Res, 91(12): e55-64, 2002). Eyes with AMD have lower levels of eNOS and of nNOS, when compared with aged control eyes, indicating that eyes with AMD have lower NO levels (BHUTTO, I. A., BABA, T., MERGES, C., MCLEOD, D. S., LUTTY, G. A. Low nitric oxide synthases (NOSs) in eyes with age-related macular degeneration (AMD). Exp Eye Res, 90(1): 155-67, 2010). Therefore PnPP-19 could act positively in early, intermediate, and late “dry” AMD, by increasing nNOS expression levels in the ocular nerve, restoring physiological levels of NO, improving vasodilatation, flow, and drusen removal. Also, it is known that oxidative stress has a key role in the pathogenesis of AMD, and there are reports on the literature of NO-donors employed to alleviate this stress (PITTALÀ, V., FIDILIO, A., LAZZARA, F., PLATANIA, C. B. M., SALERNO, L., FORESTI, R., DRAGO, F., BUCOLO, C. Effects of Novel Nitric Oxide-Releasing Molecules against Oxidative Stress on Retinal Pigmented Epithelial Cells. Oxid Med Cell Longev. 2017:1420892, 2017). PnPP-19 could also act on the reduction reaction, since the NO generated by PnPP-19-induced expression of nNOS would lead to the activation of heme oxygenase 1 (HO-1), also known as heat shock protein 32 (HSP32), one of the components of cellular defense against oxidative stress-mediated injury (FORESTI, R., CLARK, J. E., GREEN, C. J., MOTTERLINI, R. Thiol compounds interact with nitric oxide in regulating heme oxygenase-1 induction in endothelial cells. Involvement of superoxide and peroxynitrite anions, The Journal of Biological Chemistry, 272(29): 18411-18417, 1997).

### PnPP-19 in the Treatment of Diabetic Retinopathy (DR)

Diabetic retinopathy (DR) is a major complication of type 2 Diabetes Mellitus and a leading cause of vision loss in working-age populations. Clinically, DR is divided into two stages: non-proliferative DR and proliferative DR. The non-proliferative DR is the early stage of the disease, wherein increased vascular permeability and capillary occlusion are the two main clinical observations in the retinal vasculature. In this stage, retinal pathologies such as microaneurysms, hemorrhages, and hard exudates can be detected by fundus photography, even when the patients are asymptomatic. The disease eventually progresses to the proliferative manifestation, characterized by neovascularization, and severe vision impairment when the abnormal new vessels bleed into the vitreous (vitreous hemorrhage). The most common cause of vision loss in patients is diabetic macular edema (DME), which can occur at any stage of DR, causing distortion of visual images and a decrease in visual acuity. DME is a swelling or thickening of the macula due to sub and intraretinal accumulation of fluid in the macula due to the breakdown of the blood-retinal barrier. However, the disease also has an important neurodegenerative component, that includes neural apoptosis of ganglion, amacrine, and Müller cells, as well as inflammatory glial activation, and altered glutamate metabolism (WANG, W. and LO, A. C. Y. Diabetic Retinopathy: Pathophysiology and treatments. Int J Mol Sci, 19(6): 1816, 2018; BARBER, A. J. A new view of diabetic retinopathy: A neurodegenerative disease of the eye. Progress in Neuro-psychopharmacology & Biological Psychiatry, 27(2): 283-290, 2003; LYNCH, S. K., ABRÀMOFF, M. D. Diabetic Retinopathy is a neurodegenerative disorder. Vision Research, 139: 101-107, 2017).

The inducible form of HO-1 is highly expressed in the retina of diabetic rats, which led to the understanding in the literature that increased levels of HO-1 are a probable response to diabetes, with long term diabetes leading to reduced levels of HO-1 in the RPE (CUKIERNIK, M., MUKHERJEE, S., DOWNEY, D., CHAKABARTI, S., Heme oxygenase in the retina in diabetes. Current Eye Research, 27(5): 301-308, 2003; STOCKER, R. Induction of haem oxygenase as a defense against oxidative stress. Free Radical Research Communications, 9(2): 101-112, 1990; COSSO, L., MAINERI, E. P., TRAVERSO, N., ROSATTO, N., PRONZATO, M. A., COTTALASSO, D., MARINARI, U. M., ODETTI, P. Induction of heme oxygenase 1 in liver of spontaneously diabetic rats. Free Radical Research, 34(2): 189-191, 2001; DA SILVA, J. L., STOLTZ, R. A., DUNN, M. W., ABRAHAM, N. G., SHIBAHARA, S. Diminished heme oxygenase-1 mRNA expression in RPE cells from diabetic donors as quantitated by competitive RT/PCR. Current Eye Research, 16(4): 380-386, 1997). Therefore, the use of PnPP-19 for the treatment of non-proliferative DR could lead to a restoration of physiological levels of NO, reduce or eliminate the nerve damage to the optic nerve through a normalization of HO-1 levels, and a more powerful antioxidant response, leading to preserved cells and a more physiological redox environment, as well as improvements in blood flow and removal of glycating agents.

### PnPP-19 in the Treatment of NAION

Ischemic optic neuropathy is the most common acute optic neuropathy in older patients, with an annual incidence estimated at 2.3 to 10.2 cases per 100,000 persons 50 years of age or older. It can be classified as arteritic, caused by small vessels vasculitis, or non-arteritic (NAION), not caused by vasculitis (BIOUSSE, V. and NEWMAN, N. J. Ischemic Optic Neuropathies. N Engl J Med, 372:2428-2436, 2015).

NAION is caused by the the ischemia of the anterior protion of the optic nerve, particularly the lamina cribrosa, the same localion as glaucoma. The ischemia of the head of the optic nerve should be associated with a “disc at risk”, some anormality that increase the risk of nerve damage, as anatomical anormalities, optic-nerve drusen and papilledema. Hipertension, diabetes, hypercholesterolemia, stroke, ischemic heart disease, tobacco use, systemic aterosclerosis and hypercoagulability are some diseases related with NAION (BIOUSSE, V. and NEWMAN, N. J. Ischemic Optic Neuropathies. N Engl J Med, 372:2428-2436, 2015).

Up to now there is no treatment approved for NAION. The study “Ischemic Optic Neuropahty Decompression Trial (IONDT) demonstrated that when NAION occurs, the permanent visual impairment persist, but 43% of patients worse the vision loss within 6 months. Moreover, the risk of involvement of the contralateral eye is 12 to 15% (BIOUSSE, V. and NEWMAN, N. J. Ischemic Optic Neuropathies. N Engl J Med, 372:2428-2436, 2015).

NO has some beneficial effects to protect and treat NAION. NO is a vasodilator and therefore acting against ischemia; NO can down regulate the NMDA receptor (the glutamate biding receptor), and therefore reduce the excitoxicity; NO can act as a scavenger, consuming free radicals, and therefore reducing the oxidative stress. PnPP-19 can reach the retina, the local of the ischemia of the head of the optic-nerve, and increase the local levels of NO. Therefore, PnPP-19 has the potential to be the first drug useful for NAION patients.

### Peptide Synthesis

The peptides of the present description can be prepared by any methodologies known by those skilled in the art, including recombinant and non-recombinant methods. Synthetic pathways (non-recombinant) include, without limitation, the chemical synthesis of the peptide in solid phase, the chemical synthesis of the peptide in liquid phase and biocatalyzed synthesis. In a preferred embodiment, the peptides are obtained by chemical synthesis, in the liquid or solid phase, using manual, automated or semi-automated systems.

Solid-phase peptide synthesis (SPPS), for example, is known and widely employed since the description by Merrifield (MERRIFIELD, R. B. Solid Phase Peptide Synthesis. I. The Synthesis of a Tetrapeptide. J. Am. Chem. Soc., 85(14): 2149-2154, 1963). A range of variations of SPPS is available to those skilled in the art (see GUTTE, B. Peptide Synthesis, Structures, and Applications. Academic Press, San Diego, Calif., Chapter 3, 1995; and CHAN, W. C. Fmoc Solid Phase Peptide Synthesis: A Practical Approach. Oxford University Press, Oxford, 2004; MACHADO, A., LIRIA, C. W., PROTI, P. B., REMUZGO, C., MIRANDA, T. M. Sinteses quimica e enzimática de peptideos: principios básicos e aplicações. Quim. Nova, 5:781-789 2004). Briefly, the construction of the peptide by SPPS occurs in the sense C→N terminus. For that purpose, the C-terminal amino acid of interest is coupled to a solid support. The amino acid to be attached subsequently has the N-terminal portion protected with a group Boc, Fmoc or another adequate protective radical while the C-terminal portion is activated with a standard coupling reagent. Subsequently, the free terminal amine of the amino acid bound to the support reacts with the terminal carboxy portion of the subsequent amino acid. The terminal amine of the dipeptide is then deprotected and the process is repeated until the polypeptide is completed. Whenever adequate, the starting amino acids can also have protections in the side chains.

Alternatively, the peptides of the present description may be obtained by a recombinant method. Without limiting possible methodological variations, an exemplificative protocol includes: construction of the nucleic acid that encodes the peptide of interest; cloning of the said nucleic acid in an expression vector; transformation of a host cell (cells, vegetable, bacteria, such as Escherichia coli, yeasts, such as Saccharomyces cerevisiae, or mammal cells, such as Chines Hamster Ovary Cells) with the said vector; expression of the nucleic acid to produce the peptide of interest. Methods for production and expression of recombinant polypeptides in vitro and in prokaryotic and eukaryotic host cells are known to those skilled in the art (see U.S. Pat. No. 4,868,122, and SAMBROOK, J., FRITSCH, E. F., MANIATIS, T. Molecular Cloning: A Laboratory Manual. Ed. 2. Cold Spring Harbor Laboratory Press, 1989).

### Correlated Peptides

Ocular drug delivery is challenging, due to the presence of several static, dynamic and metabolic barriers. A topically applied drug that has to be delivered into the anterior eye chamber has to cross the cornea, a tri-layer tissue composed by an outer epithelium, with tight junctions connecting the most superficial cells, a central connective tissue, made of highly organized collagen, and an endothelium, mainly involved in the maintenance of the correct corneal hydration. As a result of its structure, the permeability of the cornea is low and the diffusion of drugs, particularly hydrophilic and of high molecular weight, such as peptides, is very difficult (PESCINA, S., OSTACOLO, C., GOMEZ-MONTERREY, I. M., SALA, M., BERTAMINO, A., SONVICO, F., PADULA, C., SANTI, P., BIANCHERA, A., NICOLI, S. Cell penetrating peptides in ocular drug delivery: State of the art. J Control Release. 2018 Aug. 28; 284:84-102).

Those that are skilled in the art acknowledge that certain modifications may be made on peptides such as those described in the present description, causing small or no alterations to the properties of the said peptides. Therefore, peptides related to those demonstrated herein include analogues and/or derivatives that retain some or all of the therapeutic activity of the original peptides. In this context, the term “analogue” indicates variants obtained by substitutions, deletions or additions of amino acids to the peptides described herein; while “derivative” indicates variants containing chemical modifications on the primary sequence of the peptides described herein and/or their analogues. In certain aspects, such variants may evidence improvements in at least one of the therapeutic activities of the peptides. Additionally, the peptides of the present description may be comprised of L-amino acids, D-amino acids or a combination of both in any ratio.

Another embodiment includes prodrugs or drug precursors that are chemically or enzymatically converted into any of the active peptides before, after or during the administration to a patient in need thereof. Such compounds may include among others esters, N-alkyl, phosphates or conjugates of amino acids (ARNAB, D. E., Application of Peptide-Based Prodrug Chemistry in Drug Development; Springer, New York Heidelberg Dordrecht London, 2013), more lipophilic peptides (CACCETTA, R., BLANCHFIELD, J. T., HARRISON, J., TOTH, I., BENSON, H. A. E. Epidermal Penetration of a Therapeutic Peptide by Lipid Conjugation; Stereo-Selective Peptide Availability of a Topical Diastereomeric Lipopeptide. International Journal of Peptide Research and Therapeutics, 12 (3), 327-333. 2006) and, in some cases, more hydrophilic by adding polar linkers (such as, for example, by esterification of the C-terminal domain).

Another embodiment also includes any cyclic peptide able to be converted into the linear active peptide. It further includes chemical modification with bioconjugates or macromolecules such as glycosylation or pegylation (HUTTUNEN, K. M., RAUNIO, H., RAUTIO, J. Prodrugs—from Serendipity to Rational Design. Pharmacol Rev, 63:750-771, 2011).

Another embodiment includes a peptidomimetic approach using any of the active peptides as a support to project active structures based on bioesters of groups of amino acids (VAGNER, J., QU, H. and HRUBY, V. J. Peptidomimetics, a synthetic tool of Drug Discovery. Curr Opin Chem Biol, 12(3): 292-296. 2008).

Desirable amino acid conservatives substitutions can be determined by those skilled in the art using routine methodologies. Natural amino acids may be classified in terms of the side chains properties of the as: nonpolar (glycine (Gly), alanine (Ala), valine (Val), leucine (Leu), isoleucine (Ile), methionine (Met)); uncharged polar (cysteine (Cys), serine (Ser), threonine (Thr), proline (Pro), asparagine (Asn), glutamine (Gln); acid (aspartic acid (Asp), glutamic acid (Glu)); basic (histidine (His), lysine (Lys), arginine (Arg)); and aromatic (tryptophan (Trp), tyrosine (Tyr), phenylalanine (Phe)). Exchange of amino acid for another one of the same class produces variants with functional and chemical characteristics similar to those of the original peptide. This type of modification also encompasses substitutions by artificial and/or non-essential amino acid residues, including peptidomimetics and other atypical forms of amino acids that can be regularly used during the synthesis of the peptide.

Strategies for defining substitutions of amino acids can be guided by the hydropathicity index of the side chains. The importance of hydropathic amino acids on the function of a polypeptide is understood by a person skilled in the art (KYTE, J. and DOOLITTLE. R. F. A simple method for displaying the hydropathic character of a protein. J. Mol. Biol. 157:105-31. 1982). Each amino acid has a hydropathicity index determined based on characteristics of hydrophobicity and charge. These are Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gln (−3.5); Asp (−3.5); Asn (−3.5); Lys (−3.9); and Arg (−4.5). Those skilled in the art understand that amino acids with similar hydropathicity indexes can be interchanged without significant loss of biological activity.

It is known that conservative substitutions can also be based on hydrophilicity. The average hydrophilicity of a polypeptide, determined by the hydrophilicity of the adjacent amino acids, is correlated with the biological properties of the compound. According to the patent U.S. Pat. No. 4,554,101, the natural amino acids have the following hydrophilicity values: Arg (+3.0); Lys (+3.0); Asp (+3.0±1); Glu (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); and Trp (−3.4).

In another aspect of the description, the NOS-inducer peptide comprises multimers of the active peptide which are linked by a linking group and are converted in the sole active peptide or show the pharmaceutical activity as an entire molecule (HUTTUNEN, K. and RAUTIO, J. Prodrugs—An Efficient Way to Breach Delivery and Targeting Barriers. Current Topics in Medicinal Chemistry, 11: 2265-2287, 2011). Insertions of amino acids also comprise linkers of amino acids, fusion peptides and permeation-enhancing sequences that may be added to the N-terminal or C-terminal regions of the peptides described herein. Peptides sequences able to enhance the cellular permeation and/or transcutaneous absorption are known by those skilled in the art and may be found, for example, in Kumar et al. (KUMAR, S., NARISHETTY, S. T., TUMMALA, H. Peptides as Skin Penetration Enhancers for Low Molecular Weight Drugs and Macromolecules. In: Dragicevic N., Maibach H. (eds) Percutaneous Penetration Enhancers Chemical Methods in Penetration Enhancement. Springer, Berlin, Heidelberg. 2015) and in the patents U.S. Pat. No. 14,911,019 and WO02012064429.

Cell-penetrating peptides of particular interest for improving the delivery of therapeutic peptides into the eye are known for those skilled in the art (PESCINA, S., OSTACOLO, C., GOMEZ-MONTERREY, I. M., SALA, M., BERTAMINO, A., SONVICO, F., PADULA, C., SANTI, P., BIANCHERA, A., NICOLI, S. Cell penetrating peptides in ocular drug delivery: State of the art. J Control Release. 2018 Aug. 28; 284:84-102). Also known as protein translocation domains (PTD), membrane translocation sequences or Trojan horse peptides, these sequences typically range from 5-40 amino acids (aa). Cell-penetrating peptides (CPPs) can pass through tissues and membranes of prokaryotic and eukaryotic cells, via energy-dependent or energy-independent mechanisms with no interaction with specific receptors. In general, CPPs are classified in cationic, amphipathic and hydrophobic.

Cationic CPPs have highly positive net charges at physiological pH, primarily from arginine (Arg) and lysine (Lys) residues. The CPPs belonging to this class include but are not limited to TAT derived peptides, penetrating, polyarginines, and Diatos peptide vector 1047 (DPV1047, Vectocel). Amphipathic CPPs contain both polar (hydrophilic) and non-polar (hydrophobic) regions of amino acids. Besides Lys and Arg, which are distributed throughout the sequence, they are also rich in hydrophobic residues such as Val, Leu, Ile, and Ala, A. The amphipathic CPP class include, among others, proline-rich CPP, pVEC, ARF(1-22), BPrPr(1-28), MPG, and PEP-1. Hydrophobic CPPs predominantly contain non-polar amino acids, resulting in a low net charge. This family of peptides could translocate across lipid membranes in an energy-independent manner. The class of hydrophobic CPPs includes but is not limited to gH 625, CPP-C, PFVYLI, Pep-7, and SG3 (PESCINA, S., OSTACOLO, C., GOMEZ-MONTERREY, I. M., SALA, M., BERTAMINO, A., SONVICO, F., PADULA, C., SANTI, P., BIANCHERA, A., NICOLI, S. Cell penetrating peptides in ocular drug delivery: State of the art. J Control Release. 2018 Aug. 28; 284:84-102).

In certain aspects, the above-mentioned linkers of amino acids, fusion peptides, and the permeation-enhancing sequences may have 5 to 40 additional amino acids and can be connected to the NO-inducer peptide by means of linking moieties. Such moieties may be an atom or a collection of atoms optionally used to link a therapeutic peptide to another therapeutic peptide. Alternatively, the connector molecule may consist of a sequence of amino acids designed for proteolytic cleavage in order to allow the release of the biologically active portion in an appropriate environment. Additionally, the smooth muscle tone modulating peptides described here may be fused to peptides designed to improve pharmacological properties (pharmacokinetic and/or pharmacodynamic) and or physicochemical properties.

In another aspect of the description, the NOS-inducer peptide can contain chemical modifications with one or more methyl or another small alkyl group in one or more positions of the peptide chain. Examples of such groups include methyl, ethyl, propyl, isopropyl, butyl, isobutyl, pentyl, etc. Alternatively, the NOS-inducer peptide modification results from the attachment of one or more glycosidic moieties to the peptide sequence. For example, the cited derivatives may be obtained by the attachment of one or more monosaccharides, disaccharides or trisaccharides to the peptide sequence at any position. The glycosylation may be directed to native amino acids of the peptide, or alternatively, one amino acid can be substituted or added to receive the modification.

The said glycosylated peptides may be obtained by way of routine SPPS techniques, in which the glycol-amino acids of interest are prepared prior to the synthesis of the peptide and subsequently added to the sequence in the desired position. Therefore, smooth muscle tone modulating peptides may be glycosylated in vitro. In this case, the glycosylation may occur previously. Documents U.S. Pat. No. 5,767,254, WO 2005/097158 and Doores et al., (DOORES, K., GAMBLIN, D. P. AND DAVIS, B. G. Exploring and exploiting the therapeutic potential of glycoconjugates. Chem. Commun., 12(3): 656:665, 2006), incorporated herein for the sake of reference, describes the glycosylation of amino acids. As an example, the alpha or beta selective glycosylation of residues of serine and threonine may be achieved using the Koenigs-Knorr reaction and the methodology of anomerization in situ of Lemieux using intermediary Schiff bases. The deprotection of the glycosylated Schiff base is then conducted in slightly acid conditions or by means of hydrogenolysis.

Among the monosaccharides that can be introduced into one or more residues of amino acids of the peptide described herein are glucose (dextrose), fructose, galactose, and ribose. Other monosaccharides potentially adequate for use are glyceraldehydes, dihydroxyacetone, erythrose, threose, erythrulose, arabinose, lyxose, xylose, ribulose, xylulose, allose, altrose, mannose, N-Acetylneuraminic acid, fucose, N-Acetylgalactosamine, N-Acetylglucosamine, among others. Glycosides, such as mono-, di- and trisaccharides for use in the modification of the PnPP-19 may be of synthetic or natural origin. Disaccharides that can be introduced into one or more residues of the amino acids described herein include sucrose, lactose, trehalose, alose, melibiose, cellobiose, and others. The trisaccharides can be acarbose, raffinose, and melezitose.

In an additional aspect of some embodiments, the NOS-inducer peptide herein can be modified such that there occurs only a partial reduction or no reduction of the biological activities and properties of the said peptide. In some cases, such modifications can be realized to result in an improvement of the intended therapeutic activity. Therefore, the scope of some embodiments of the present invention includes variants that retain at least 1%, 5%, 10%, 20%, 30%, 40%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99%, and any range derivable therefrom such that, for example, at least 70% to at least 80%, and preferably at least 81% until 90%, or yet more preferably, between 91% and 99% of the therapeutic activity relatively to the non-modified peptide. The scope of some embodiments of the present invention also includes variants that have a therapeutic activity higher than 100%, 110%, 125%, 150%, 200% or more than 300%, or yet that evidence a 100 or 100 times higher activity, and any range derivable therefrom, in comparison with the non-modified peptides.

The NOS-enhancer peptide described in some embodiments of the present invention may also be covalently conjugated to hydrosoluble polymers, either directly or by means of a spacer group. Examples of peptide-polymer conjugates inserted in the scope of some embodiments of this invention include: conjugates containing a hydrosoluble polymer coupled to the peptide in a detachable or stable manner, particularly coupled to the N-terminal portion; conjugates containing a hydrosoluble polymer coupled to the peptide in a detachable or stable manner, particularly coupled to the C-terminal portion; conjugates containing a hydrosoluble polymer coupled to the peptide in a detachable or a stable manner, particularly coupled to an amino acid located internally in the peptide chain; conjugates containing more than one hydrosoluble polymer coupled to the peptide in a detachable or a stable manner, coupled to the peptide in distinct regions such as, for example, to the N-terminal portion and to the side chain of an amino acid located internally in the peptide sequence (particularly a lysine). Alternatively, an amino acid, to which the hydrosoluble will be coupled may be inserted in the N-terminal or C-terminal portions, or in the middle of the primary structure of the peptide.

Typically, the above-contemplated polymer is hydrophilic, non-peptidic, biocompatible and non-immunogenic. In this respect, a substance is deemed biocompatible if the beneficial effects associated with the administration thereof to living organisms, either alone or combined with another substance (for example, a biologically active ingredient such as a therapeutic peptide), overcomes any deleterious effect that is clinically observable. A substance is deemed non-immunogenic if the intended use of the substance in vivo does not produce an undesirable immunological response (for example, the formation of antibodies) or, if an immunological response is triggered, such event is not deemed clinically significant or important. Example of such hydrosoluble polymers include, without limitation: polyethylene glycol (PEG), polypropylene glycol (PPG), copolymers of ethylene glycol and propylene glycol, polyolefinic alcohol, polyvinylpyrrolidone, poly(hydroxyalkyl methacrylamide), poly(hydroxyalkyl methacrylate), sulfated of non-sulfated polysaccharides, polyoxazolines, poly(N-acryloyl morpholine), and combinations of these polymers, including copolymers and terpolymers thereof.

The above-cited hydrosoluble polymers are not limited to a particular architecture and may have linear of no-linear structures, such as branched, bifurcated, multi-branched (for example, PEGs coupled to a polyol core), or dendritic (densely branched structure with several terminal groups). Methods for the conjugation of polymers to peptides are described in the prior art, as well as the adequate reagents, which may be selected among alkylating or acylating agents (see HARRIS, J. M. and ZALIPSKY, S., Poly(ethylene glycol), Chemistry and Biological Applications. ACS, Washington, 1997; VERONESE, F., and HARRIS, J. M. Peptide and Protein PEGylation. Advanced Drug Delivery Reviews, 54(4); 453-609. 2002; ZALIPSKY, S., LEE, C. Use of Functionalized Poly(Ethylene Glycols) for Modification of Polypeptides. in Polyethylene Glycol Chemistry: Biotechnical and Biomedical Applications, J. M. Harris, ed., Plenus Press, New York, 1992; ZALIPSKY, S. Functionalized poly(ethylene glycol) for preparation of biologically relevant conjugates. Advanced Drug Reviews, 16:157-182, 1995; and in ROBERTS, M. J., BENTLEY, M. D., HARRIS, J. M., Chemistry for peptide and protein PEGylation. Adv. Drug Delivery Reviews, 54, 459-476, 2002). Typically, the average molecular weight of hydrosoluble polymers may vary between 100 Daltons (Da) and 150,000 Da (150 kDa). For example, there may be used hydrosoluble polymers with an average molecular weight of 250 Da to 80 kDa, from 500 Da to 65 kDa, from 750 Da to 40 kDa, or 1 kDa to 30 kDa.

In an additional aspect of some embodiments of the invention, the NOS-inducer peptide may be acylated in one or more positions of the peptide chain in order to improve physicochemical, pharmacokinetic and/or pharmacodynamic characteristics. For example, the introduction of lipophilic acyl groups is widely employed to increase the plasma half-life of therapeutic peptides, since they render the groups coupled thereto less susceptible to oxidations. Methods and reagents for acylation of peptides are known to those familiar with the art. Documents WO 98/08871, US 2003/0082671, WO 2015/162195, incorporated herein as references, exemplify reagents and conditions for acylation of peptides. The modification of free amines with acyl groups is particularly useful to promote the acylation of peptides and proteins (ABELLO, N., KERSTJENS, H. A., POSTMA, D. S., BISCHOFF, R. Selective acylation of primary amines in peptides and proteins. Journal of proteome research, 6(12): 4770-4776. 2007). In this particular case, the NOS-inducer peptide may be acylated at the N-terminal amine or in the side chain of one or more amino acids originally present in the sequence or inserted for the purpose of receiving the acylation in question.

In one aspect of some embodiments of the present invention, there are provided pharmaceutical compositions comprising the peptide of some embodiment of the present invention. In a particular modality, the peptide of the present invention is combined with another active pharmaceutical ingredient (API). In a further aspect, an embodiment of the peptide of the invention, alone or combined with another API, is further combined with pharmaceutically acceptable vehicles and/or excipients and/or additives.

### Formulation

The pharmaceutical compositions of the invention can be prepared and formulated in accordance with the conventional methods such as disclosed, for example, in the British, European and United States Pharmacopoeias (British pharmacopoeia. Vol. 1. London: Medicines and Healthcare products Regulatory Agency; 2018; European pharmacopoeia. 9th ed, Strassbourg: Council of Europe: 2018; United States Pharmacopoeia, 42, National Formulary 37, 2018), Remington's Pharmaceutical Sciences (REMINGTON, J. P., AND GENNARO, A. R. Remington's Pharmaceutical Sciences. Mack Publishing Co., 18th ed. 1990), Martindale: The Extra Pharmacopoeia (MARTINDALE, W. AND REYNOLDS, J. E. F. Martindale: The Extra Pharmacopoeia. London, The Pharmaceutical Press 31st ed, 1996), Harry's Cosmeticology (HARRY, R., and ROSEN, M. R. Harry's cosmeticology. Leonard Hill Books, 9th ed. 2015), and in Prista's Pharmaceutical technology (PRISTA, L. V. N., ALVES, A. C., MORGADO, R. M. R. Técnica Farmacêutica e Farmácia Galênica. 4th ed. Fundação Calouste Gulbenkian. Serviço de Educação e Bolsas, 1996).

Pharmaceutical compositions can be formulated for any route of administration including, for example, topical, oral, nasal, rectal or parenteral administration. The term parenteral, as used herein, includes subcutaneous, intradermic, intravascular (for example, intravenous), intramuscular, spinal, intracranial, intrathecal and intraperitoneal injections, as well as any similar technique of injection or infusion. However, in some embodiments, the administration in the eye is a preferred embodiment of this invention. Ocular administration of the peptides may be carried out topically using, for example, eye drops, or by intracameral, intrastromal, subconjunctival, intravitreal or subchoroidal injections. In some embodiments, topic administration is a preferred embodiment of this invention. Topic administration with eye drops is a further preferred embodiment.

Topical ophthalmic forms (such as eye drops) are sterile and can be liquid, semi-solid, or solid preparations, that may contain one or more active pharmaceutical ingredient(s) intended for application to the conjunctiva, the conjunctival sac or the eyelids. The different categories of ophthalmic preparations include drops consisting of emulsions, solutions or suspensions, and ointments. The vast majority of aqueous ocular dosage forms are solutions. Suspensions may be required whenever the therapeutic agent exhibits problems regarding chemical stability, or to increase the potency of lipophilic drugs (that is greater compared to when in water-soluble salts).

The choice of drug salt is important as the local ocular application of certain drug salts results to improve the solubility (physicochemical properties in solution) and reduce pain/irritation or stinging. Usually, the concentration of the drugs in eye drops is high to compensate for the poor retention. For PnPP-19, the main choice of salts is acetate, chloride, bromide, carbonate, phosphate, palmitic acid, caproic acid or histidine.

The manufacture of ophthalmic product needs to consider several important aspects related to the main features of the eye, as the local of permeation. Conjunctiva has a large surface area (circa 18 cm), helps to produce and to maintain the tear film and has greater permeability to the diffusion of therapeutic agents compared to the cornea. The cornea controls the diffusion of drugs into the inner chambers of the eye by lacrimal fluid, and it is non-vascular and negatively charged. Therefore in order to permeate cornea therapeutic agents must exhibit intermediate solubility in both lipid and aqueous phases and must be of low molecular weight. Other important aspects that interferes with the pharmacokinetic and therefore efficacy of ophthalmic product include but are not limited to the following: (i) vehicle and concentration; (ii) pH and buffering; (iii) tonicity; (iv) viscosity; (v) clarity; (vi) additives; (vii) preservatives; (viii) sterility; (ix) aseptic filling; (x) packaging of the finished product.

Vehicle and Concentration. A vehicle that is predominantly used for the formulation of aqueous ocular dosage forms is Purified Water USP. Water for injections is not a specific formulation requirement. Occasionally, an oil can be used if the therapeutic agent is profoundly unstable within an aqueous vehicle. The choice of oils for ocular use is similar to that for parenteral use. The concentration of the therapeutic agent has to follow manufacture, must lie within 95-105% of the nominal concentration. Over the shelf-life of the product, the concentration of drug must not fall below 90% of the nominal amount. The concentration of the drug has to consider the absorption across the cornea, the successful treatment of glaucoma using ocular formulations requires that there is sufficient drug absorption across the cornea. To be effectively absorbed the drug must exhibit differential solubility, i.e. the ionized and non-ionized forms coexist; sufficient concentration of the non-ionized form is required to partition into and diffuse across the lipid-rich outer layer of the cornea (the epithelium). The inner layer of the cornea (the stroma) is predominantly aqueous and therefore ionization of the drug must occur to enable partitioning into this phase. Following diffusion to the interface of between the stroma and the endothelial (lipid-rich) layer, absorption of the non-ionised (but not the ionized) form occurs. The non-ionized drug then diffuses to the endothelium/aqueous humour interface where ionization and dissolution into the aqueous humour occur. The pKa of the therapeutic agent determines the ionization of the therapeutic agent at defined pH values. To overcome this problem, the appropriate form of the acid salt is one that the pH of the solution is acidic and stability is optimized. The addition of buffer allows the formulation to be instilled into the eye, and the lacrimal fluid adjusts the pH to physiological conditions, thereby facilitating absorption.

pH: Ideally, the pH of the ocular solution should be controlled at 7.4 as this is the pH of tear fluid. However, the choice of pH of the formulation is also dictated by the stability of the therapeutic agent at that pH, which in turn serves to define the shelf-life of the formulation; and whether (or not) absorption of the active agent across the cornea is required. The pH of PnPP-19 formulations ranges from 4.5 to 7.4, preferably from 5.6 to 7.4, more preferably from 6.6 to 7.4.

Buffers: Solution pH/inclusion of buffers. The pH and the control of the pH of ocular formulations are important determinants of the stability of the therapeutic agent, the ocular acceptability of the formulation and the absorption of the drug across the cornea. Ideally, the pH of the formulation should be one that maximizes the chemical stability (and, if required, absorption) of the therapeutic agent. This issue is particularly important due to the effect of pH on the stability of peptides. As highlighted in the previous section, the pH and the buffer capacity directly affect the subsequent discomfort of the formulation.

Tonicity: The formulation is isotonic or more preferably hypotonic when considering the human eye pH. Tonicity pH of the lacrimal fluid is 7.4 and is isotonic with blood. This fluid possesses a good buffer capacity (due to the presence of carbonic acid, weak organic acids, and protein), being able to neutralize unbuffered formulations effectively over a wide range of pH values (3.5-10.0). The main tonicity modifier use in ocular forms is sodium chloride. Typically ocular aqueous dosage forms are not specifically formulated to be isotonic (0.9% w/w NaCl equivalent) and may be formulated within a range of tonicity values equivalent to between 0.7% and 1.5% w/w NaCl.

Viscosity: The rate of turnover of lacrimal fluid is approximately 1 μl/min and the blinking frequency in humans is circa 15-20 times per minute. These physiological functions act to remove the therapeutic agent/formulation from the surface of the eye. A viscosity-increasing agent improves the time of contact of the compound and the corneal surface, which reduces the removal by the lacrimal fluid. The viscosity-increasing agent may be present in a concentration of from 0.05% to 0.5% w/w, and more preferably from 0.3 to 0.4% w/w. Viscosity-modifying (enhancing) agents are hydrophilic polymers that are added to ocular solutions for two main reasons: (i) to control the rate at which the drop flows out of the container (and thus enhance ease of application); and, more importantly, (ii) to control the residence time of the solution within the precorneal environment. For example, it has been shown that the retention of an aqueous solution within the precorneal region is short (frequently less than 1 minute); however, if the viscosity is increased, the retention may be enhanced. Furthermore, it has been reported that there is a critical formulation viscosity threshold (circa 55 mPa/s) above which no further increase in contact time between the dosage form and the eye occurs. It must be remembered that there is an upper limit below which the viscosity of eye drop formulations must be maintained as this may lead to blockage of the lacrimal ducts. The viscosities of commercially available products are frequently lower than 30 mPa/s. The enhancement of the viscosity of ocular suspensions will serve to enhance the physical stability of ocular suspensions. Ideally the viscosity-modifying agent should exhibit the following properties: (i) easily filtered: all eye drop solutions are filtered during the manufacturing process; (ii) easily sterilized: sterilization of eye drop solution is usually performed by filtration or by heat (the viscosity-modifying agent must be chemically and physically stable under these conditions); (iii) compatible with other components and the therapeutic agent: the interaction of hydrophilic polymers with certain preservatives is well-known. The viscosity-increasing agent is typically a polymeric compound, for example, carbomers or cellulose-based polymers. Preferably the polymer is a carbomer, carboxymethylcellulose, hydroxyethylcellulose, ethylcellulose, methylcellulose, sodium or hydroxypropylmethylcellulose (HPMC, such as Hypromellose USP). Hydroxypropylmethylcellulose (HPMC) in aqueous ocular formulations HPMC is used in the concentration of 0.45-1.0% w/w.
a) Poly(vinyl alcohol). This is a water-soluble vinyl polymer that is available in three grades: (i) high-viscosity (average molecular weight 200 000 g/mol); (ii) medium-viscosity (average molecular weight 130 000 g/mol); and (iii) low-viscosity (average molecular weight 20 000 g/mol). It is used to enhance the viscosity of ocular formulations in concentrations ranging from 0.25% to 3.00% w/w (the actual concentration is dependent on the molecular weight of the polymer used).
b) Poly(acrylic acid). This is a water-soluble acrylate polymer that is cross-linked with either allyl sucrose or allyl ethers of pentaerythritol. It is predominantly used in ocular aqueous formulations for the treatment of the dry-eye syndrome. However, it may be used to increase the viscosity of ocular formulations that contain a therapeutic agent.

Clarity. This may be simply to remove any particles (e.g. clarification using a 0.8-μm filter) or clarification in conjunction with filtration and sterilization.

Additives. PnPP-19 can be formulated with antioxidants, surfactants and/or polymers. Antioxidants may be added to ocular solutions/suspensions to optimize the stability of therapeutic agents that degrade by oxidation. Sodium metabisulphite (circa 0.3%) is an example of an antioxidant that is commonly used for this purpose. Surfactants (anionic, cationic) agents are predominantly employed in aqueous suspension to enhance the physical stability of the dispersed particles; and to solubilize therapeutic agents in aqueous ocular solutions. One of the primary concerns regarding the use of surfactants agents in ocular dosage forms is the potential toxicity/irritancy. Accordingly, non-ionic surfactants are preferentially (and predominantly) used whereas the anionic surfactants on ocular solution/suspension dosage forms are avoided. Polymers can be natural or synthetic.

Preservatives: Preservatives are antimicrobial agents that usually are included in formulations unless the active ingredient itself has antimicrobial activity. Ophthalmic preparations supplied as multidose preparations may include a suitable antimicrobial agent. Ocular formulations should be sterile and the antimicrobial activity should remain effective throughout the entire period of use. An ideal preservative is rapidly effective and topically non-irritating. It may be a single antimicrobial agent or a mixture of such agents. Common preservatives used with ophthalmics include:
a) benzalkonium chloride (BAK, 0.002% to 0.02% w/v (typically 0.01% w/v) and benzethonium chloride (0.01 to 0.02% w/v). The antimicrobial properties of benzalkonium chloride decrease whenever the pH of the formulation falls below 5.0; they are incompatible with anionic therapeutic agents and also with non-ionic hydrophilic polymers used for viscosity. Cationic preservatives as 0.1% w/v disodium edetate (disodium EDTA), commonly used in ocular solutions, can be included in ocular formulations in which benzalkonium chloride is used to enhance its antimicrobial activity by chelating divalent cations in the outer membrane of the bacterial cell.
b) Parabens. Mixtures of methyl and propyl esters of para-hydroxybenzoic acid are used in ophthalmic formulations (typically at a combined concentration of 0.2% w/w). There is a concern regarding the ocular irritancy of the parabens, which limits their use in ophthalmic preparations. This problem is augmented by the need to increase the concentration of parabens in ocular formulations that contain hydrophilic polymers, due to the interaction between these two species.
c) Organic mercurial compounds. These are antimicrobial agents that contain mercury and, due to environmental and toxicity concerns, are not commonly used in ocular formulations nowadays. The main examples are phenylmercuric acetate, phenylmercuric nitrate (which is sometimes supplied as a mixture with phenylmercuric hydroxide) and thimerosal. The concentration ranges of the antimicrobial agents used in ocular formulations are: 0.001 to 0.002% w/v for phenylmercuric acetate, 0.002% w/v for phenylmercuric nitrate and 0.001 to 0.15% w/v and 0.001 to 0.004% w/v thimerosal (when used in ophthalmic solutions and suspensions, respectively). The phenylmercuric salts have been reported to be deposited in the lens of the eye (termed mercuria lentis) when formulated in preparations that are designed for chronic usage, e.g. for the treatment of glaucoma. Thimerosal is not associated with this problem; however, it has been associated with ocular sensitization. As a result, these preservatives are only used in ocular formulations whenever there is no suitable option.
d) Organic alcohols. Chlorobutanol and phenylethylalcohol are examples of organic alcohols. Chlorobutanol can be used at a concentration of 0.5% w/v. Hydrolysis of chlorobutanol occurs under alkaline condition, and HCl is liberated as a by-product (the rate of reaction increases with increasing temperature, e.g. during autoclaving). The use of chlorobutanol is reserved for acidic ophthalmic preparations, Chlorobutanol is volatile and may be lost from the solution if stored in polyolefin containers, due to partitioning. Accordingly, preparations that employ this preservative must be stored in glass containers. One final problem associated with the use of chlorobutanol is its limited solubility. Phenylethylalcohol shares similar problems, e.g. poor solubility, volatility and partitioning into plastic containers. The typical concentration used in ophthalmic preparations is 0.25-0.50% v/v.
e) Others. Potassium sorbate, chlorhexidine acetate, chlorocresol, and polyhexamine gluconate also can be used as preservatives.

Sterility. The formulation should be sterile.

Packaging of the finished product. The formulations according to some embodiments the invention are preferably packaged in end-use containers of suitable material, most preferably are comprised of a sterile single-dose unit container for easy administration or multi-dose containers. The material needs to be compatible with the formulations and does not permit the formulations to degrade or the components to permeate through the material. Optionally, the container will be constructed to protect the contents from light, for example using colored or opaque materials, and may also be enclosed in further packaging for this purpose. Suitable materials include plastics such as polyethylene terephthalate, polypropylene or preferably high density polyethylene (HDPE), preferably in ampoules made predominantly of high density polyethylene (HDPE), and most preferably in a squeezable HDPE container to provide a single dose, in unit doses ranging from 2 to 100 ml, preferably in unit doses of 5 to 50 ml, and most preferably in unit doses of 5, 10 and 50 ml. Thus, containers containing from about 2 to 7 ml, e.g. 5 ml, or about 7 to 12 ml, e.g. 10 ml, or about 25 to 35 ml, e.g 30 ml, or about 45 to 55 ml, e.g. 50 ml of the formulation are preferred. The container may be itself either a preformed sterile ampoule that is filled and sealed, or it may be formed, filled and sealed in one process (Blow-Fill-Seal technology). A container may be fitted with an aerosol spray head and contain a formulation according to some embodiments of the invention whereby the formulation can be delivered as an aerosol spray. A further aspect of some embodiments of the invention is that the unit dose containers are constructed in such a manner that the seal on the dispensing mouth can be reattached after opening to prevent spillage or contamination. The formulations and the containers in which they are packaged are also preferably such that they can be used directly in the eye.

Extended-release for optical application are formulations such as a capsule, pill or coated table that diminishes and/or delays the release of the active ingredient(s) after administration in the eye. Formulations with controlled release may be administered, for example through an implant in a target location. In general, a formulation with controlled release may be obtained by means of the combination of the active ingredient(s) with a matrix material that, in itself, changes the release rate and/or through the use of a coating with controlled release, which delays the disintegration and absorption in the location of implant, and thereby provides a delayed or a sustained action during a longer period. One type of formulation with the controlled release is a formulation with sustained release, in which at least one active ingredient is continuously released during a period of time at a constant rate. Preferably, the therapeutic agent is released at a rate such that the concentrations in the blood (for example, plasma) are maintained within the therapeutic range, however below the toxic levels, during a period of time that is at least 4 hours, preferably at least 8 hours, and more preferably at least 12 hours. Preferably, a formulation provides a constant level of release of the modulator. The amount of modulator contained in a formulation with sustained-release depends, for example, on the location of the implant, the expected release the rate and duration and the nature of the condition to be treated or prevented. Such formulations may, in general, be prepared using well-known technologies. Formulations may have vehicles that may be biocompatible and/or biodegradable.

The release rate may be varied using methods well known in the art including (i) variation of thickness of composition of the coating, (ii) alteration of the quantity of manner of addition of plasticizer on a coating, (iii) inclusion of additional ingredients, such as agents that modify the release, (iv) alteration of the composition, particle size or format of particle of the matrix and (v) provision of one or more passages through the coating. The amount of modulator contained within a sustained release formulation depends, for example, from the method of administration (for example, the location of the implant), the rate and duration of release that is expected and the nature of the condition to be treated or prevented.

The matrix material, which may or not have a controlled release function, is generally any material that supports the active ingredient(s). For example, a material such as a glyceryl monostearate or glyceryl diesterate may be employed. Active ingredient(s) may be combined with the matrix material prior to the formation of the dosage form (for example, an eye drop). Alternatively, or furthermore, the active ingredient(s) may be coated on the surface of a particle, granule, sphere, microsphere, globule or pellet that comprises the matrix material. Such coating may be obtained via conventional means, such as through dissolution of the active ingredient(s) in another adequate solvent and spraying. Optionally, extra ingredients are added prior to the coating (for example, to aid in the binding of the active ingredient(s) to the matrix material).

### Combination Therapy

In some embodiments of the present invention, the compositions can comprise, in addition to the NOS-inducer peptide of the present invention, one or more additional APIs. Compared with non-fixed combinations, fixed-combination glaucoma therapies provide various demonstrated benefits, a reduction of exposure to preservatives and lower risk of preservative-related ocular surface disease symptoms, and a reduced number of total administrations. Further, due to simplification of the administration regimen, fixed combinations may improve treatment adherence and persistence, thereby improving stability of IOP control over time (HOLLÓ, G., VUORINEN, J., TUOMINEN, J., HUTTUNEN, T., ROPO, A., PFEIFFER, N. Fixed-dose combination of tafluprost and timolol in the treatment of open-angle glaucoma and ocular hypertension: comparison with other fixed-combination products. Adv Ther, 31(9):932-44. 2014).

Particular fixed-combinations of some embodiments of the present invention involve the NOS-inducer peptide of the present invention and prostaglandin analogs, β-adrenergic antagonists, α-adrenergic agonists, acetylcholine receptors agonists, carbonic anhydrase inhibitors, and Rho kinase (ROCK) inhibitors. Less preferred, but also useful combinations comprise the NO-inducer peptide of the present invention and PDE5 inhibitors, steroidal and non-steroidal anti-inflammatory drugs, and anti-histamine agents.

### Correlation Between Animal Models and Humans

It is important to communicate the pedigree of models along with the results that they yield. Pedigree would consider factors such as the extent to which the model is based on a well-established theoretical framework (SCANNELL, J. W., and BOSLEY, J. When Quality Beats Quantity: Decision Theory, Drug Discovery, and the Reproducibility Crisis. PLoS One. 11(2): e0147215, 2016). Here, we report the activity of PnPP-19 on preclinical models of glaucoma. A method of treating human patients is then derived from the results produced in animals.

Translatability of preclinical data to clinical settings largely depends on how predictable the animal models are. In turn, predictability is a function of construct validity, formally defined as the degree to which the set of features of an experiment represents the features of an intended entity. In preclinical research, construct validity has often been used to describe the relationship between the functional features in animal models (e.g. etiology of disease, onset and progression, symptoms, treatment schedules and routes of administration, and outcomes) and the disease intended to treat in humans (HENDERSON, V. C., KIMMELMAN, J., FERGUSSON, D., GRIMSHAW, J. M., HACKAM, D. G. Threats to validity in the design and conduct of preclinical efficacy studies: a systematic review of guidelines for in vivo animal experiments. PLoS Med, 10(7): e1001489, 2013).

Henderson et al (2013) reviews the literature on preclinical research guidelines and outlines the recommendations to improve construct validity of preclinical experiments, namely: (i) matching model to human manifestation of the disease; (ii) characterization of animal properties at baseline; (iii) matching time of treatment delivery to the expected clinical setting; (iv) matching the route of administration to the desired clinical application; (v) determine the pharmacokinetics; (vi) matching outcome measurement to the clinical setting; (vii) verify the treatment response according to the mechanistic pathway; (viii) assess multiple manifestations of the disease phenotype; (ix) use validated assays to assess molecular pathways; (x) address confounds associated with the experimental setting. These recommendations guided the design of the examples further described.

A successful hypertensive glaucoma model should induce structural glaucomatous changes: including loss of retinal nerve fibers, retinal ganglion cells and optic-disc cupping along with IOP elevation. The level and duration of IOP elevation should be titratable depending on the targeted glaucomatous damage.

Particularly in the development of new drugs to treat glaucoma, there is relevant information in the literature using animal models to demonstrate the IOP-lowering ability of the drugs currently being employed in the treatment of glaucoma. Although there are some anatomical differences (table 1), at least with regard to medications aimed at lowering IOP, ocular hypertensive animal models of glaucoma are better able to identify those drugs that will go on to commercial availability (CHAN, C. Animal Models of Ophtalmic Diseases. Springer, Cham. 2016). The models with the highest predictive power regarding the human disease are those that achieve RGC degeneration via experimental elevation of IOP.

TABLE 1
Anatomical eye differences between
most used animals models and human.

| Eye feature | Rat | Rabbit | Human |
|---|---|---|---|
| Corneal thickness (mm) | ≈.21 | ≈0.4 | ≈0.55 |
| AH vol (mL) | 0.009-0.023 | ≈0.25 | 0.24-0.28 |
| IOP (mmHg) | 11-15 | 13-22 | 13-18 |
| Outflow rate (μL/min) | 0.119 | 2.8-3.7 | 1.5-3.0 |
| Conventional outflow (%) | ≈20 | ≈92 | 60-90 |

Rabbits are sensitive to most IOP lowering agents, with the exception of prostaglandins analogs (WOODWARD, D. F., BURKE, J. A., WILLIAMS, L. S., PALMER, B. P., WHEELER, L. A., WOLDEMUSSIE, E., RUIZ, G., CHEN, J. Prostaglandin F2 alpha effects on intraocular pressure negatively correlate with FP-receptor stimulation. Invest Ophthalmol Vis Sci, 30(8): 1838-42, 1989; ORIHASHI, M., SHIMA, Y., TSUNEKI, H., KIMURA, I. Potent reduction of intraocular pressure by nipradilol plus latanoprost in ocular hypertensive rabbits. Biol Pharm Bull, 28(1): 65-8, 2005). One glaucoma model can be achieved by injecting hypertonic saline into the vitreous body, which leads to rapid IOP rise in these animals. This ocular hypertension can be significantly blunted by treatment with latanoprostene bunod (a prostaglandins analogs associated with NO donor), but not with latanoprost (prostaglandins analogs alone) (KRAUSS, A. H., IMPAGNATIELLO, F., TORIS, C. B., GALE, D. C., PRASANNA, G., BORGHI, V., CHIROLI, V., CHONG, W. K., CARREIRO, S. T., ONGINI, E. Ocular hypotensive activity of BOL-303259-X, a nitric oxide donating prostaglandin F2α agonist, in preclinical models. Exp Eye Res, 93(3):250-5, 2011). In dogs, more specifically beagles inherited POAG was described in 1981 and remains a highly used glaucoma model (GELATT, K. N., GUM, G. G., GWIN, R. M. Animal model of human disease. Primary open angle glaucoma. Inherited primary open-angle glaucoma in the beagle. American Journal of Pathology, 102(2): 292-295, 1981; BOUHENNI, R. A., DUNMIRE, J., SEWELL, A., EDWARD, D. P. Animal models of glaucoma. J Biomed Biotechnol, 2012:692609, 2012). Glaucomatous dogs treated with latanoprostene had a reduction in IOP of 27%, and glaucomatous dogs treated with latanoprostene bunod had a reduction in IOP of 44% (KRAUSS, A. H., IMPAGNATIELLO, F., TORIS, C. B., GALE, D. C., PRASANNA, G., BORGHI, V., CHIROLI, V., CHONG, W. K., CARREIRO, S. T., ONGINI, E. Ocular hypotensive activity of BOL-303259-X, a nitric oxide donating prostaglandin F2α agonist, in preclinical models. Exp Eye Res, 93(3):250-5, 2011). Those models are adequate to test new drugs that act on the NO pathway, because latanoprostene bunod (an association of NO donor and prostaglandin), is more effective than latanoprost (prostaglandin alone).

There are several induced rodent hypertensive glaucoma models including intracameral injection of microbeads, laser photocoagulation, episcleral vein cauterization, injection of hypertonic saline and hyaluronic acid (HA) (BISWAS, S., WAN, K. H. Review of rodent hypertensive glaucoma models Acta Ophthalmol, 97(3), 2019).

Injection of HA induces glaucoma in rats. Although the HA model requires reintervention, it consistently maintains elevated IOP up to 62 days. In rodents, this model has a longer-lasting effect of a single injection of HA due to the shallower anterior chamber in rats such that the actual intracameral concentration of HA is higher than in other species, added with an incomplete washout of the HA in rats. The excess deposition of HA can avoid the normal outflow of aqueous humour (AH) by reducing the diameter of corneoscleral intertrabecular spaces and/or regulating the aqueous flow through the juxtacanalicular basement membrane. With repeated HA injections, the hypertensive condition can be extended to 10 weeks, resulting in a 40% RGC loss (BENOZZI, J., NAHUM, L. P., CAMPANELLI, J. L., ROSENSTEIN, R. E. Effect of hyaluronic acid on intraocular pressure in rats. Invest Ophthalmol Vis Sci, (7):2196-200, 2002).

In general, drug candidates that reduced diurnal IOP by 19% and produced a peak reduction of 26% or more in those types of hypertensive glaucoma models eventually reached the market, while the non-commercialized drugs produced average peak reductions of 15% (STEWART, W. C., MAGRATH, G. N., DEMOS, C. M., NELSON, L. A., STEWART, J. A. Predictive value of the efficacy of glaucoma medications in animal models: preclinical to regulatory studies. Br J Ophthalmol, 95(10):1355-60, 2011). The anatomical differences between animals and humans may explain little discrepancies, as for example the length of time the pressure is reduced in an animal model is often less than in the human (STEWART, W. C., MAGRATH, G. N., DEMOS, C. M., NELSON, L. A., STEWART, J. A. Predictive value of the efficacy of glaucoma medications in animal models: preclinical to regulatory studies. Br J Ophthalmol, 95(10):1355-60, 2011) (Table 2).

TABLE 2
Comparison of IOP average reduction in the HA hypertensive
glaucoma rat model with human glaucoma of the most
used glaucoma drugs, including latanoprostene bunod
and netasurdil that work on NO pathway.

| | Average IOP reduction (%) | |
|---|---|---|
| Selected therapies | HA-induced glaucoma in rats | Human glaucoma |
| Timolol | 25.3 | 18 |
| Latanoprost | 28.1 | 13.1 |
| Latanoprostene Bunod | N/A | 31 |
| Netarsudil | 23* | |

*Not done in a model of HA-induced glaucoma, instead, the model employed was of optical nerve crushing.

The methods to prevent and treat eye diseases related to intraocular hypertension and/or optic nerve degeneration of some embodiments of the present invention can be derived from the following examples in light of the foregoing discussion.

### EXAMPLES

### Synthesis of PnPP-19

The NO-inducer peptide of the present invention was chemically synthesized by Fmoc/t-buyl synthesis in solid support, in resin Rink-amide (0.68 mmol/g) produced by the company GenOne (Rio de Janeiro, Brazil). The final cleavage and deprotection were realized with water-TFA-1.2-ethanedithiol-triisopropyl silane, 92.5-2.5-2.5-2.5 (v/v), 25° C., 180 min. The peptides were extracted with an aqueous solution of 50% (v/v) of acetonitrile and purified by reverse-phase chromatography (RPC) in a column of Sephasil C8 peptide (5 μST 4.6/100-HPLC), balanced with water TFA 0.1%. The samples were eluted using a gradient of acetonitrile with 0.1% de TFA, a flow rate of 2 ml/min, at 280 nm. Furthermore, the peptide was N-terminally acetylated and C-terminally amidated.

### HET-CAM System, Ocular Irritation Model

Hen's egg—chorioallantoic membranes (HET-CAM) were used to assess anti-irritant properties of different concentrations of PnPP-19. PnPP-19 was dissolved in 300 μL of saline in order to achieve the concentration of 40 μg, 80 μg, 160 μg, 320 μg, in 20 μL (the volume of one eye drop). Sodium chloride 0.9% (NaCl) was used as negative control and sodium hydroxide 0.1 M (NaOH) as a positive control.

The appearance and intensity of any reactions were observed at 0 s, 30 s, 2 min and 5 min. The reactions were classified according to a semi-quantitatively analysis, on a scale from 0 (no reaction) to 3 (strong reaction). The ocular irritation index (OII) was then calculated by the following expression:

$$OII = \frac{( 301 - h ) \times 5}{300} + \frac{( 301 - l ) \times 7}{300} + \frac{( 301 - c ) \times 9}{300}$$

where h is time (in seconds) from the beginning of bleeding; l lysis, and c coagulation. The following classification was used: OII≤0.9: slightly irritant; 0.9<OII≤4.9: moderately irritant; 4.9<OII≤8.9: irritant; 8.9<OII≤21: severely irritant.

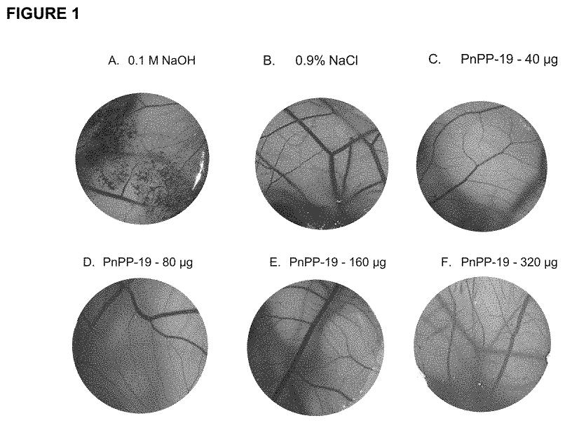

The results demonstrated that the positive control, 0.1 M NaOH, causes initial lesions in the first 30 s, such as bleeding and rosette-like coagulation (FIG. 1A). The mean cumulative score of the positive control (0.1 M NaOH) was 21.11±0.32, demonstrates that this control is adequate since it is severely irritant (Table 3).

The negative control and PnPP-19 in all concentrations tested did not show any signs of vascular response (FIG. 1B-F), and the mean cumulative scores calculated for the test were ≤0.9 which ranked PnPP-19 as non-irritant (Table 3).

TABLE 3
Ocular Irritation Index (OII) scores for the test. The score
was calculated according to the equation mentioned above and the
corresponding classification. The results are expressed as mean ±
S.D. (n = 6). NI = Non- irritant; SI = severely irritant.

| Tested solution | OII ± SD | Irritability classification |
|---|---|---|
| 0.1M NaOH (Positive control) | 21.11 ± 0.32 | SI |
| 0.9% NaCl (Negative control) | ≤0.9 ± 0.0 | NI |
| PnPP-19 - 40 μg | ≤0.9 ± 0.0 | NI |
| PnPP-19 - 80 μg | ≤0.9 ± 0.0 | NI |
| PnPP-19 - 160 μg | ≤0.9 ± 0.0 | NI |
| PnPP-19 - 320 μg | ≤0.9 ± 0.0 | NI |

### In Vivo Single-Dose Acute Toxicology

Single-dose topical application of PnPP-19 was dissolved in 300 of saline at the dose of 40, 80 and 160 μg, in 20 μL of saline, in the form of eye drops, was evaluated in normotensive Wistar rats (150-200 g), n=4 rats per dose group. Fundoscopy and electroretinography were performed at days 0, 1, 7 and 15 after PnPP-19 administration. At day 15, eye tissue was collected to histopathological analysis. Day 0 was considered as a control time point for any dose to evaluate for observed differences.

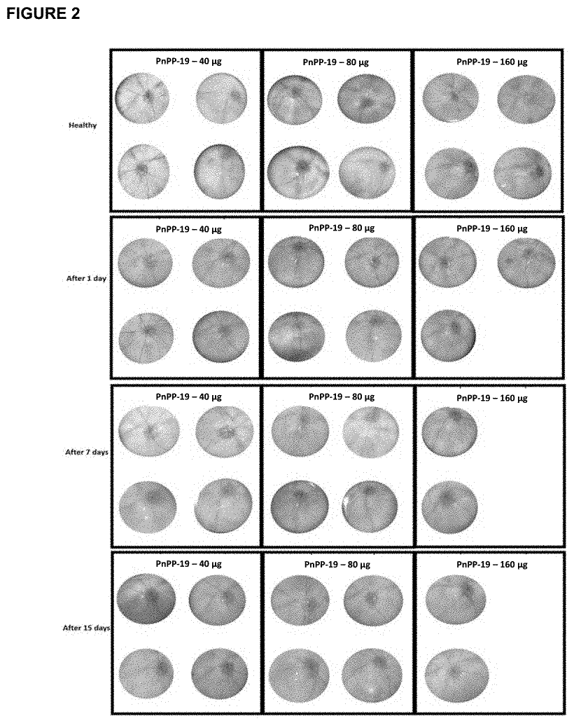

No difference was observed regarding fundoscopy between day 0 and the subsequent days (FIG. 2). No pallor or excavation, neither bleeding and drusen were detected in any treatment groups for up to 15 days.

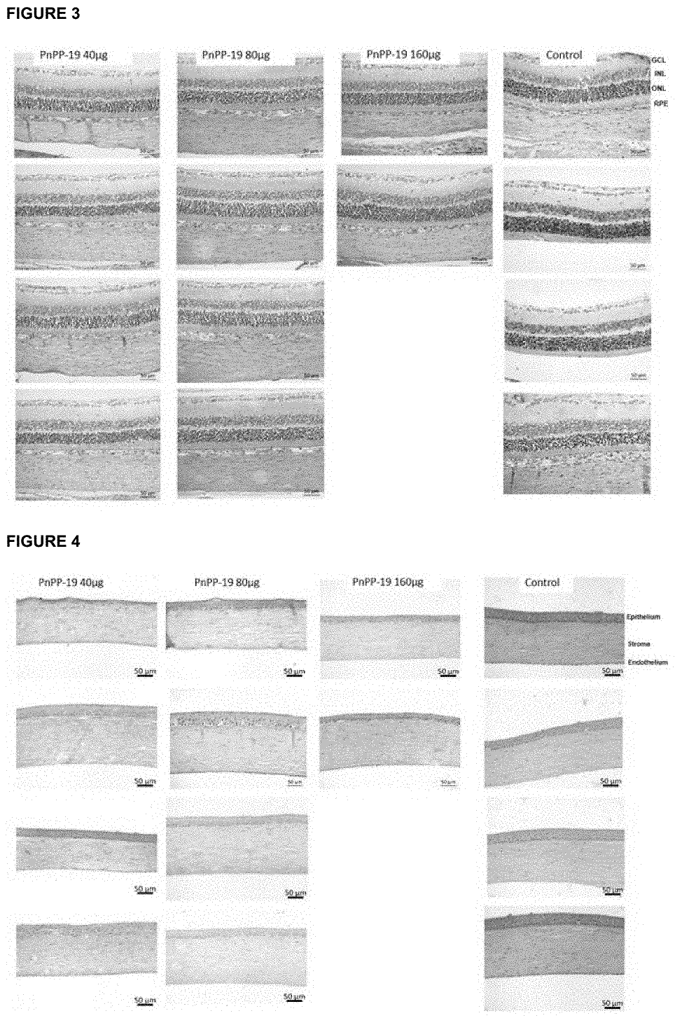

Histological analysis of the cornea and retina demonstrated that the PnPP-19 treated groups keeps the same morphology compared to day 0 (control) at any dose (FIGS. 3 and 4).

Electroretinography showed the maintenance of the curve shape of a and b wave in all treatment groups, compared to day 0 (control) (data not shown), demonstrating the absence of retinal nerve damage at any tested dose of PnPP-19.

### Modulation of IOP in Healthy Rats

PnPP-19 efficacy was evaluated in vivo after topical administration of 80 μg of the peptide in 20 μL in saline 0.9% in male normotensive Wistar rats (150-200 g), n=8. The IOP measurements were performed using a TonoPen (Tono-PenVet, Reichert) in baseline and after PnPP-19 administration in the lower conjunctival sac of the left eye of animals. The right eye was used as a control (saline administration). To perform the measurements, unsedated animals were topically anesthetized by the instillation of 0.5% proxymetacaine hydrochloride. Three IOP readings (with SE less than 5%) were taken for each eye. The average of these three readings was considered the corresponding value of the IOP. The IOP measurements were performed in time points of 1, 2, 3, 4, 5, 6 h after PnPP-19 single-dose administration. The % of IOP reduction was calculated as demonstrated by the following equation:

$$IOP decrease ( \% ) at scheduled time = \frac{{IOP}_{before administration} - {IOP}_{at scheduled time}}{{IOP}_{before administration}} \times 100 ( \% )$$

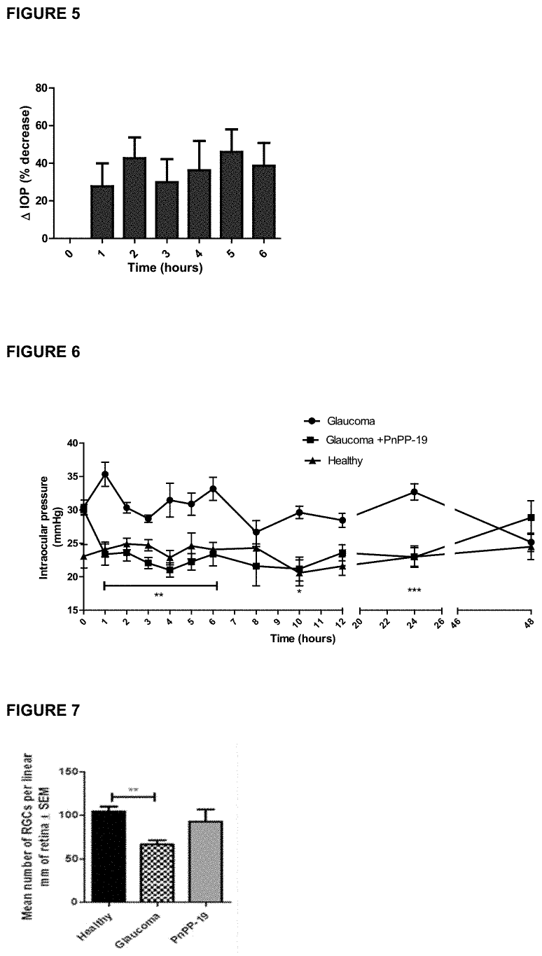

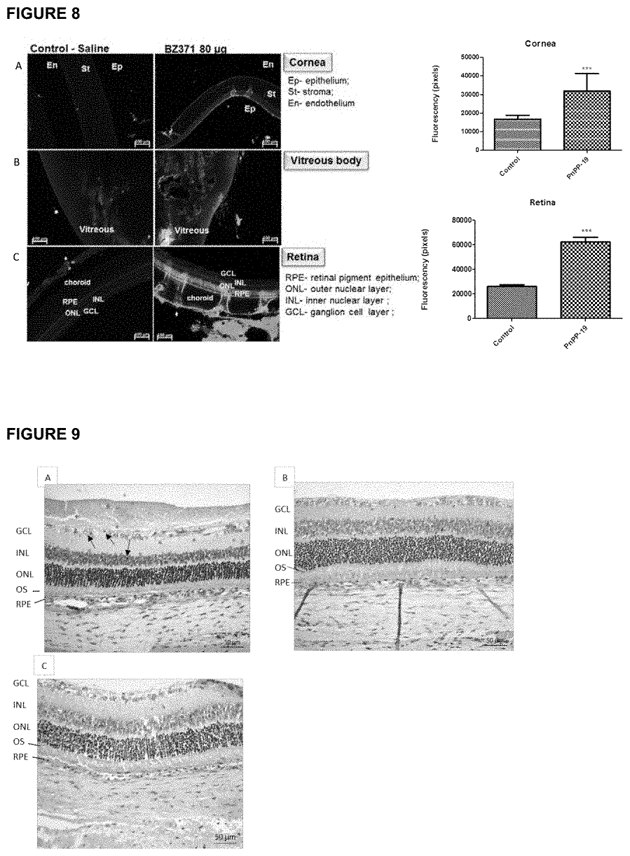

IOP reduction after a single dose application of PnPP-19 (80 μg/20 μL) was: 19.08±2.29 mmHg, after 2 hours of treatment, compared to control, 23.25±2.06 mmHg; 18.20±2 mmHg, after 4 hours of treatment, compared to control, 22.95±4.35 mmHg; 17.16±2.13 mmHg, after 5 hours of treatment, compared to the control 22.5±2.13 mmHg. This data was then expressed as % of IOP reduction (Δ). PnPP-19 reduced IOP by 40, 36 and 45% after 2, 4 and 5 hours of administration, respectively. This reduction was maintained for up to 6 hours (FIG. 5).

### Modulation of IOP in Hypertensive Models in Glaucoma

The effectiveness of the PnPP-19 was first evaluated by measuring the changes in IOP of glaucomatous male Wistar rats (180-220 g, 7 to 10 weeks old, n=6). Glaucoma was induced in the right eye by the injection of 30 μL hyaluronic acid (HA) (10 mg/mL) into the anterior chamber through the clear cornea once per week for 3 weeks on the same calendar day and at the same time. Evaluation of the IOP was performed using a TonoPen (Tono-PenVet, Reichert). To perform the measurements, unsedated animals were topically anesthetized by the instillation of 0.5% proxymetacaine hydrochloride. Three IOP readings (with SE less than 5%) were taken for each eye. The average of these three readings was considered the corresponding value of the IOP. Electroretinogram (ERG) was recorded before and after the glaucoma induction. Eyes were enucleated and corneas and retinas were prepared for histology. The groups of treatment were defined as Healthy or non-glaucomatous—left eye, where HA was not applied; Glaucomatous, Non-treated—right eye, where HA was applied, treated with control (vehicle, saline);

Glaucomatous, treated with PnPP-19—right eye, where HA was applied, treated with PnPP-19 at 80 μg/20 μL (one eye drop) in the eye; IOP lowering with conventional eye drop was maintained during 24 hours after the treatment. We found that after the establishment of ocular hypertension, PnPP-19 treated group had lower IOP than the glaucomatous non-treated group at 24 hours of instillation of the treatments (22.9±3.6 vs 29.4±3.5 mmHg, respectively, p<0.001), similar to the healthy group (FIG. 6).

The effects of PnPP-19 on rat's vision at the end of the HA glaucomatous model was analyzed by ERG to evaluate if the peptide impacts the visual acuity. Dark-adapted ERG records were performed before the treatment and 72 hours after the PnPP-19 treatment instillation. It was observed differences in the pattern of ERG curves comparing healthy eyes with glaucomatous eyes. There was no difference between the glaucomatous non-treated group with PnPP-19 treated groups (data not shown).

Histologic analysis showed that the glaucomatous rats exhibited an increase in the excavation of the optic nerve due to the severe loss of the neural fibers of RGCs and to a large reduction in neural fibers of the optic nerve. Treatment with PnPP-19 reduced this histologic damage if compared to untreated glaucoma group.

Relative to control healthy retinas, the glaucomic non-treated retinas showed a decrease in cell number, and increases in cytoplasmic vacuolization and the number of pyknotic nuclei in the ganglion cell layer (GCL). The inner nuclear layer (INL) displays more edema, pyknotic nuclei, and cellular disorganization. The outer nuclear layer (ONL) exhibits a decreased cell number, and greater edema and cell disorganization compared with control retinas.

The loss of the RGCs, cells susceptible to ischemia, was detected in the glaucomatous non-treated animals compared to healthy animals. PnPP-19 preserves the number of RGCs: glaucomatous animals treated with PnPP-19 have an RGC count higher than untreated glaucomatous rats and was not statistically different compared to healthy rat (66.6±12.5 cells vs. 93.3±34.6 cells, respectively, p<0.01) (FIG. 7).

### In Vivo Pharmacokinetic—Eye Permeation and Diffusion of PnPP-19

Fluorescence-labeled PnPP-19 (FITC) was applied topically in form of eye drops (peptide dissolved in saline) in a dose of 80 μg/20 μl in the male normotensive Wistar rat eye. Vehicle (saline) was applied in the contralateral eye to be used as control. After 3 hours the eyes were enucleated and prepared for histological analysis using fluorescence microscope APOTOME.2 ZEISS.

Fluorescence was more detected in the eyes were the FITC PnPP-19 was applied, compared to vehicle, in the cornea, vitreous body and retina. This data demonstrated that labeled PnPP-19, in a simple saline solution, permeates from the cornea to retinal epithelium within 3 hours after application.

### Neuroprotective Effect of PnPP-19 in Ischemic Retina Model

Retinal ischemia is very common in many ocular disorders such as age-related macular degeneration (AMD), diabetic retinopathy, retinal vascular occlusion, or glaucoma. Retinal ischemia induces irreversible morphological and functional changes that may result in blindness. Previous reports demonstrated that ischemia/reperfusion (I/R) in the optic nerve in a rodent model leads to morphological and functional changes of different retinal cell types, specifically, loss of RGCs and amacrine cells. Other changes include optic nerve damage, neuronal degeneration, a tissue dissolution, structural distortion, and increased microglial activation. (RENNER, M., STUTE, G., ALZUREIQI, M., REINHARD, J., WIEMANN, S., SCHMID, H., FAISSNER, A., DICK, H. B., JOACHIM, S. C. Optic Nerve Degeneration after Retinal Ischemia/Reperfusion in a Rodent Model. Front Cell Neurosci, 11:254, 2017).

The potential neuroprotective effect of PnPP-19 in the retina was investigated with an animal model of ischemic injury. For this purpose, male Wistar rats were treated with PnPP-19 either before or after induction of ischemia (80 μg in 20 μl (one eye drop) once a day administration for seven days).

Male Wistar rats (180-200 g) were anesthetized intraperitoneally with ketamine hydrochloride 90 mg/kg and xylazine hydrochloride 10.0 mg/kg and the retinas were made ischemic according to the protocols of Hughes (HUGHES, W. F. Quantitation of ischemic damage in the rat retina. Exp Eye Res, 53(5): 573-82, 1991), and of Louzada-Junior et al. (Louzada-Junior, P., Dias, J. J., Santos, W. F., Lachat, J. J., Bradford, H. F., Coutinho-Netto, J. Glutamate Release in Experimental Ischaemia of the Retina: An Approach Using Microdialysis. J Neurochem, 59(1):358-63, 1992). IOP was elevated by cannulating the eye anterior chamber, with a sterile 27-gauge needle attached to a manometer/pump connected to an air reservoir (HUGHES, W. F. Quantitation of ischemic damage in the rat retina. Exp Eye Res, 53(5): 573-82, 1991) elevating the IOP to 155 mmHg for 40 min evoking ischemia (indicated by whitening of the eye fundus as blood flow is interrupted).

After the ischemic period, the IOP was allowed to return to normal levels for 45 min (reperfusion period, during which the fundus color returns to normal). The left retina of each animal was subjected to the experimental condition, ischemia and/or reperfusion, while the right retina served as a nonischemic control.

To assess whether PnPP-19 is able to have a neuroprotective effect in the retina and optic nerve from the damage caused by retinal ischemia, two studies were performed:

Study 1 (post-treatment)—80 μg of PnPP-19 in 20 μl of saline solution (one drop) was instilled for 7 days at the same time, after induction of retinal ischemia in the animals. The number sample was equal to 6 animals/eyes per group.

Study 2 (pre-treatment or prevention)—80 μg of BZ371 in 20 μl of saline solution (one drop) was instilled for 7 days at the same time, before the induction of retinal ischemia in the animals. The number sample was equal to 6 animals/eyes per group.

After induction of retinal ocular ischemia in all groups, retinal function was assessed by electroretinogram (ERG) 1 day and 7 days after induction of ischemia. ERGs were carried out in compliance with the International Society for Clinical Electrophysiology (ISCEV) guidelines. ERG was performed 0 and 72 hours after the PnPP-19 administration (80 μg/eye). ERGs were recorded using an Espion e2 electrophysiology system and a Ganzfeld LED stimulator (ColorDome™ desktop Ganzfeld, Diagnosys LLC, Littleton, Mass.). All ERGs were recorded after 12 h of dark adaptation. The pupils were dilated using one drop of 0.5% tropicamide (Mydriacyl; Alcon, São Paulo, Brazil) and the animals were anesthetized by intramuscular injection (ketamine hydrochloride 90 mg/kg and xylazine hydrochloride 10.0 mg/kg) before the recording of ERG. The eyes were topically anesthetized with 0.5% proxymetacaine hydrochloride (Anestalcon; Alcon, São Paulo, Brazil) immediately before the ERG recordings. Bipolar contact lenses electrode were placed on both corneas and a needle electrode was inserted into the back. Impedance was set to less than 5 kΩ at 25 Hz in each electrode. The dark-adapted (scotopic) ERG protocol was recorded according to a modified ISCEV protocol and presented in the following sequence: rod (0.01 cd.s/m2) and combined response (3 cd.s/m2) with 30 s interstimulus interval (ISI), with a duration of 4 ms.

After day 7 the animals were euthanized and the eyes were collected for histological analysis. Immediately after sacrifice, eyes were enucleated and fixed in Davidson solution (two parts of 10% neutral phosphate-buffered formalin, three parts of 95% ethanol, one part of glacial acetic acid and three parts of ultrapure water). Samples were included in paraffin and 4-μm-thick sections of the in the sagittal plane, to allow dorsal-to ventral observation of the cornea and retina were stained with hematoxylin and eosin and were analyzed in unmyelinated areas under light microscopy using a microscope (Zeiss®, Model Axio Imager M2).

Previous studies demonstrated that the changes in the retinal layer thickness accurately reflected the changes in cell number, according to Hughes' and Li reports (HUGHES, W. F. Quantitation of ischemic damage in the rat retina. Exp Eye Res, 53(5): 573-82, 1991; LI, L., WANG, Y., QIN, X., ZHANG, J., ZHANG, Z. Echinacoside protects retinal ganglion cells from ischemia/reperfusion-induced injury in the rat retina. Molecular Vision; 24:746-758, 2018). We measured different layer thicknesses to quantify the degree of cell loss to measure the ischemic damage in the rat retina. The thicknesses of the entire retina (between the inner limiting membrane and the pigment epithelium), the inner nuclear layer (INL) and the outer nuclear layer (ONL) were measured. The measurements (400×) were made 0.5 mm dorsal and ventral from the optic disc. The number of cells in the ganglion cell layer (GCL) was calculated using the linear cell density (cells per 200 μm). For each eye, three measurements at adjacent locations in each hemisphere were made. The mean of three or more eyes was recorded as the representative value for each group.

The effect of instillation of PnPP-19 after ischemic induction was analyzed by the histological changes observed when comparing ischemic/untreated (FIG. 9A), ischemic/PnPP-19 post-treatment (FIG. 9B) and healthy retinas (FIG. 9C). PnPP-19 post-treatment have similar histology compared to the healthy retinas, except by displaying a reduction in vacuolization and number of pyknotic nuclei in all layers (FIG. 9B-C). On the other hand, in the ischemia/untreated retinas the GCL showed lower cell density, and increases in vacuolization, number of pyknotic nuclei (black arrows), and cellular disorganization; the INL also had fewer cells and had more pyknotic nuclei and cytoplasmic vacuoles; and there were also fewer cells in the ONL, in comparison with healthy and ischemic/PnPP-19 post-treatment retinas (FIG. 9A-C).

The overall thickness of the retina of ischemic/PnPP-19 post-treatment group was similar to the healthy group (174.66±19.66 μm versus 175.31±14.22 μm); on the other hand, in the ischemic/untreated group, it was reduced around 30% (121.08±21.38 μm) compared to healthy and to ischemic/PnPP-19 treatment groups (p:<0.001 for both comparisons). The thickness of the INL and ONL of ischemic/PnPP-19 treatment group was similar to the healthy group (INL—31.82±3.14 μm versus 31.16±3.80 μm), (ONL—47.39±1.93 μm versus 44.98±9.47 μm); on the other hand, in the ischemic/untreated group the thickness of the INL and ONL were reduced by 20% and 28%, respectively, compared to healthy group (p<0.05 and p:<0.001, respectively) and 24% and 30% compared to ischemic/PnPP-19 treatment group (p:<0.05 and p:<0.001, respectively). The GCL number was similar between ischemic/PnPP-19 treatment and healthy groups (31.75±3.5 versus 30.0±5.5 cells per 200 μm) (Table 4). The GCL density was reduced by 35% and 31% in the ischemic/untreated group compared with ischemic/PnPP-19 treatment and healthy groups, respectively (p: <0.001 for both) (FIG. 9). This results demonstrated that PnPP-19 reduces the histological damage caused by retinal ischemia and avoid the loss of RGC.

TABLE 4
The thickness of the retinal layers and GCL cell count at 7
days after Isquemia and treatment (PnPP-19 post-treatment).

| Group | Thickness (μm) | | | GCL number |
|---|---|---|---|---|
| (n ≥ 3) | Entire retina | INL | ONL | (cells per 200 μm) |
| Healthy | 175.31 ± 14.22 | 31.16 ± 3.80 | 44.98 ± 3.47 | 30.0 ± 5.5 |
| Untreated | 121.08 ± 21.38^{aabb} | 22.21 ± 5.24^{ab} | 35.64 ± 5.79^{aabb} | 20.6 ± 2.5^{ab} |
| Treatment PnPP-19 | 174.66 ± 19.66 | 31.82 ± 3.14 | 47.39 ± 1.93 | 31.75 ± 3.5 |

Values are (mean ± SD), n ≥ 3.
Statistical significance was determined with one-way ANOVA test with Dunnett's post-test.
(a): comparison with the healthy group;
(b): comparison with the treatment group.
p < 0.05;
^{aa}or ^{bb}p < 0.001.
INL, inner nuclear layer; ONL, outer nuclear layer; GCL, ganglion cell layer.
Untreated group: high IOP-induced ischemia injury without treatment; Treatment PnPP-19 group: high IOP-induced ischemia injury with PnPP-19 treatment.

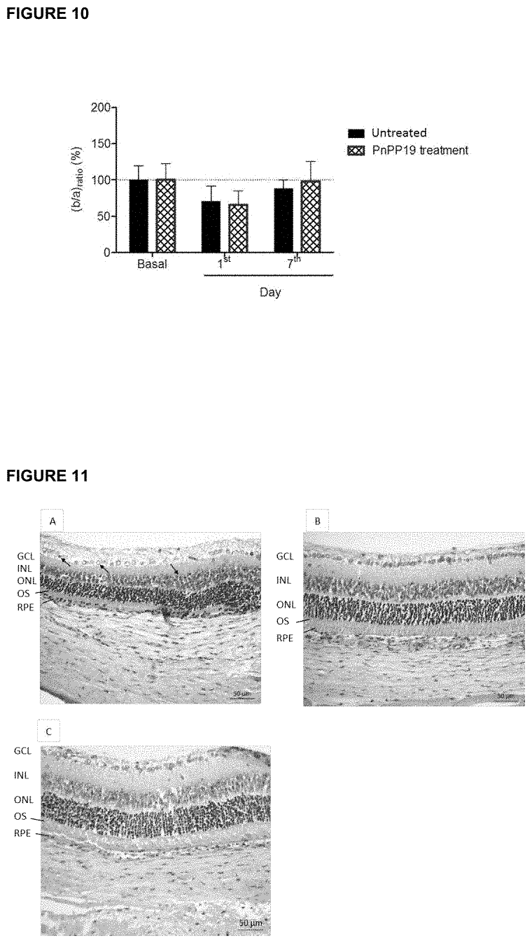

The ERG analysis of study 1 (PnPP-19 post-treatment) demonstrated differences in the pattern of ERG curves for ischemic/untreated and ischemic/PnPP-19 post-treatment eyes. Although we observe alterations in amplitude and implicit time of a- and b-waves at dark-adapted condition if compared with healthy eyes, the shape of the curves was not affected in any group. There were no significant differences between ischemic eyes receiving treatment or not. Another way to evaluate the functional activity of the retina by the ERG is to calculate the ratio (%) between the amplitudes of the a-wave and b-wave in response to the stimulus luminous 3 cd.s.m⁻² under scotopic condition compared to healthy eyes (100% for the mean value of the control group). It was possible to observe that there was not a significant difference between the untreated and PnPP-19 post-treated ischemic eyes (FIG. 10).

The effect of instillation of PnPP-19 before ischemic induction was analyzed by the histological changes observed when comparing ischemic/untreated (FIG. 11A), ischemic/PnPP-19 post-treatment (FIG. 11B) and healthy retinas (FIG. 11C). The ischemic/untreated retinas group (FIG. 11A) showed a decrease in cell number, and increases in cytoplasmic vacuolization and the number of pyknotic nuclei in the ganglion cell layer (GCL) compared to healthy retina group; in addition to that, the INL display pyknotic nuclei and cellular disorganization; the ONL exhibits a decreased cell number (table 5). In PnPP-19 pre-treatment retinas group (FIG. 11B), we observed no degeneration, pyknotic nuclei or disorganization of the cells.

The overall thickness of the retina in the ischemic/untreated group was reduced around 21% compared to the healthy group (140.68±13.37 versus 179.47±12.42 μm, p<0.001); the ischemic/PnPP-19 pre-treatment group presented with 11% higher thickness compared to ischemic/untreated group (157.12±8.43 μm versus 140.68±13.37 μm, p<0.05), and reduced around 17% compared to the healthy group, although not statistically significant (157.12±8.43 μm versus 179.47±12.42 μm) (table 5).

The thickness of the INL of the ischemic/untreated group was reduced by 22% compared to the healthy group (24.83±4.08 versus 31.91±3.94 μm, p<0.05). The thickness of the ONL, although presented with a reduction of 10%, was not statistically different from the healthy group (39.77±7.09 versus 44.40±3.70 μm). The thickness of the INL and ONL of ischemic/PnPP-19 pre-treatment group was not statistically different neither from the healthy group nor from the ischemic/untreated group (table 3).

The GCL density was reduced by 36% and 40% in the ischemic/untreated group compared to the ischemic/PnPP-19 pre-treatment and healthy group, respectively (p<0.05 for both) (Table 5, FIG. 11).

TABLE 5
The thickness of the retinal layers and GCL cell count at 1 day after Isquemia
(treatment 7 days before the ischemic injury (PnPP-19 pre-treatment).

| Group | Thickness (μm) | | | GCL cell number |
|---|---|---|---|---|
| (n ≤ 3) | Entire retina | INL | ONL | (cells per 200 μm) |
| Healthy | 179.47 ± 12.42 | 31.91 ± 3.94 | 44.40 ± 3.7 | 30.00 ± 5.50 |
| Untreated | 140.68 ± 13.37^{aab} | 24.83 ± 4.08^{a} | 39.77 ± 7.09 | 18.00 ± 4.50^{ab} |
| Treatment PnPP-19 | 157.12 ± 8.43 | 28.31 ± 1.16 | 39.51 ± 5.80 | 28.25 ± 4.50 |

Values are (means ± SD), n ≥ 3.
Values compared between groups by one-way ANOVA with Dunnett's post-test,
a: compared with the healthy group;
b: compared with the treat PnPP-19 group p < 0.05;
^{aa}or ^{bb}p < 0.001.
INL, inner nuclear layer; ONL, outer nuclear layer; GCL, ganglion cell layer.
Untreated: high IOP-induced ischemia injury without treatment; Treatment PnPP-19: high IOP-induced ischemia injury with PnPP-19 treatment.

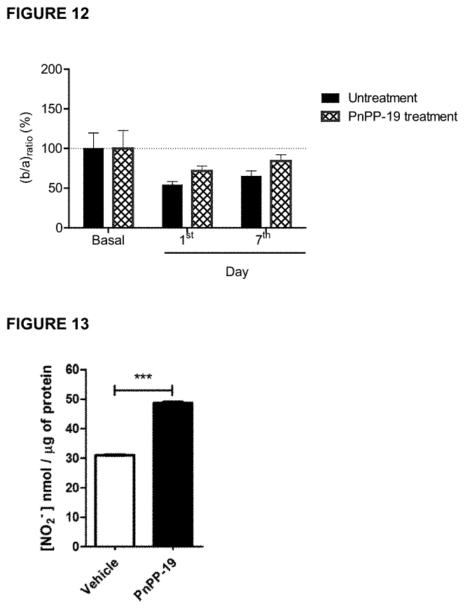

The ERG analysis of study 2 demonstrated statistically difference in implicit time of b-waves during the exposure to 0.01 and 3.0 cd.m.s⁻² in eyes healthy compared to ischemic/untreated eyes, but not with ischemic/PnPP-19 pre-treatment eyes. The ratio (%) between the amplitudes of the a-wave and b-wave in response to the stimulus luminous 3 cd.s.m⁻² under scotopic condition, showing that there was not a significant difference between the ischemic/untreated and ischemic/PnPP-19 pre-treatment eyes compared to healthy eyes; however, an increasing tendency in b/a ratio in ischemic/PnPP-19 pre-treatment retinas was observed (FIG. 12).

Overall in both study 1 and study 2, PnPP-19 treated and protected RGC against ischemic injury, indicated by a reduction in histological damage and improved the rat's vision after ischemic injury, making it closer to the values of healthy eyes.

### In Vivo Pharmacology Study of PnPP-19

NO level was indirectly determined by measuring the concentration of nitrite using the Griess methodology. PnPP-19 or vehicle was topically administered in healthy rats. After 2 hours, the animals were euthanized and their eyes were collected (N=6). Lens and retina were extracted. The remaining tissues from each animal were homogenized in 100 μL of saline.

The samples were then centrifuged at 4° C. (5000 g, 10 minutes), and 30 μL of the homogenate were applied in duplicate to a microliter plate well, followed by 30 μL of Griess reagent (0.2% [w/v] naphthyleneethylene diamine and 2% [w/v] sulfanilamide in 5% [v/v] phosphoric acid). After 10 minutes at room temperature, the absorbance was measured with a microplate reader (Tecan Infinite00 PRO, Meilen, Switzerland) at a wavelength of 540 nm. NO2-standard reference curves were made with sodium NO2-in saline at 20, 15, 12.5, 10, 5, and 1.5 μM. The detection limit of the assay was ˜1.5 μMin distilled water. The total amount of protein found in the eye tissue was estimated by NanoDrop 2000 Spectrophotometer (Thermo Scientific Madison, Wis.) and the nitrite release was normalized per μg of protein.

PnPP-19 stimulate an increase in nitrite production in healthy rat's eye tissues, compared to vehicle (48.70+/−1.19 vs 31.01+/−0.38 of nitrite nmol/mg of protein) (FIG. 13).

### Phase 1 Clinical Trial (In Humans)

Twelve healthy subjects, 6 males and 6 females, were selected to evaluate the safety and tolerability of PnPP-19. PnPP-19 were applied as one eye drop per day (with 250 μg of peptide in 50 μL saline (0.5%)) for 7 days, in one eye, while vehicle (the formulation without the peptide) was applied in the contralateral eye. The eye were randomized in a double-blind way to each intervention.

The safety was analyzed by an ophthalmologist thought a slit lamp in conjunction with a biomiscrscope to examine the anterior and posterior segment of the eye. This procedure was performed in both eyes, in each subject, in the days 1, 2, 3, 4 and 7 of the treatment. There was no safety finding. Moreover, there was no difference in blood pressure, heart rate, weight, physical examination and electrocardiogram results during the study.

The tolerability was analyzed thought a daily tolerability questionnaire. Four events were reported: one patient had puritis in the eye, mild and of very short duration, soon after the PnPP-19 was instillated in just one day; Two patients related eye burning, mild and of very short duration, soon after the PnPP-19 (in one patient) and vehicle (in the other) was instillated in just one day. One patient reported mild headache, that last minutes. The investigator considered that none of those adverse events were related to PnPP-19.

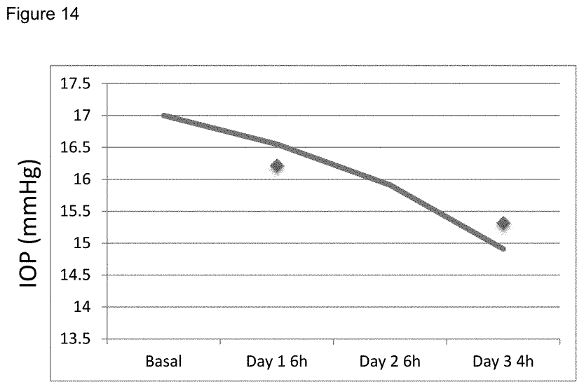

IOP was measured with a non-contact tonometer before and 1,2,4 6 and 24 hours after the instillations of PnPP-19 or vehicle. The IOP of the 3rd day of treatment was statistically lower than the IOP of the first day (FIG. 14).

### SEQUENCE LISTING

This application contains a Sequence Listing which has been submitted electronically in ASCII format (file name: 4_20200911_Seq_Listing_ST25; created: Nov. 9, 2020; Size: 4,096 bytes) and is hereby incorporated by reference in its entirety.

1. A method of reducing the intraocular pressure, comprising topically administering to an eye an effective amount of PnPP-19, as shown in SEQ ID NO: 1.

2. A method of treating or preventing ischemic optic neuropathy, comprising topically administering to an eye an effective amount of PnPP-19.

3. The method of claim 1 wherein the administration is one or two drops per day of a composition comprising, in a pharmaceutically acceptable liquid medium, PnPP-19 in an effective amount, specifically between 0.08 to 0.72% of peptide per volume.

4. The method of claim 2 wherein the ischemic optic neuropathy is glaucoma.

5. The method of claim 4 wherein the glaucoma is normal-tension glaucoma.

6. The method of claim 2 wherein the ischemic optic neuropathy is age-related macular degeneration, diabetic neuropathy or non-arteritic ischemic optic neuropathy (NAION).

7. The method of claim 1 wherein administering PnPP-19 starts before vision loss of the eye.

8. The method of claim 1 wherein administering PnPP-19 starts before partial vision loss of the eye due to intraocular pressure or ischemic optic neuropathy.

9. The method of claim 8, wherein the administering PnPP-19 continues after partial vision loss of the eye due to intraocular pressure or ischemic optic neuropathy.

10. The method of claim 1 wherein administering PnPP-19 starts after partial vision loss caused by ischemic optic neuropathy of the eye.

11. A pharmaceutical composition for ophthalmic administration, comprising, in a pharmaceutically effective medium, an effective amount of PnPP-19, specifically between 0.08 to 0.72% of peptide per volume, and one or more pharmaceutically acceptable excipients.

12. A method of treating glaucoma in a patient, comprising topically administering to an eye of a patient in need thereof, a composition formulated for ophthalmic administration comprising, in a pharmaceutically acceptable medium, an effective amount of PnPP-19 specifically between 0.08 to 0.72% of peptide per volume.

13. The method of claim 12, wherein the patient has normal intraocular tension.

14. A method of treating a patient with ocular hypertension, comprising topically administering to an eye of a patient in need thereof, a composition formulated for ophthalmic administration comprising, in a pharmaceutically acceptable medium, an effective amount of PnPP-19 specifically between 0.08 to 0.72% of peptide per volume.

15. A method for the reduction of intraocular pressure in a patient, comprising topically administering to an eye of a patient in need thereof, an effective amount of PnPP-19 specifically between 0.08 to 0.72% of peptide per volume, formulated for ophthalmic administration comprising, in a pharmaceutically acceptable medium.

16. The method of claim 14, wherein the patient has elevated intraocular pressure.

17. The method of claim 16, wherein the patient has glaucoma.
